(19) United States
(12) Patent Application Publication
(10) Pub. No.: US 2010/0015258 A1
(43) Pub. Date: Jan. 21, 2010

(54) Rice Bran Extracts and Methods of Use Thereof

(76) Inventors: Randall S. Alberte, Estero, FL (US); William P. Roschek, JR., Naples, FL (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 12/467,848

(22) Filed: May 18, 2009

Related U.S. Application Data
(60) Provisional application No. 61/054,151, filed on May 18, 2008.
(60) Provisional application No. 61/101,475, filed on Sep. 30, 2008.
(60) Provisional application No. 61/147,305, filed on Jan. 26, 2009.

(51) Int. Cl. A61K 36/899; A61P 3/10

(52) U.S. Cl. 424/750

(57) ABSTRACT

The present invention relates to stabilized rice bran extracts that modulating glucose uptake and FABP4 activities that control glucose uptake in to cells and carbohydrate and fat metabolism. These stabilized rice bran extracts are useful for treating hypoglycemia, diabetes, and obesity.

### RELATED APPLICATIONS

This application claims the benefit of priority to U.S. Provisional Application Nos. 61/054,151, filed on May 18, 2008, 61/101,475, filed on Sep. 30, 2008, and 61/147,305, filed on Jan. 26, 2009, each of which is herein incorporated by reference in its entirety.

### FIELD OF INVENTION

The present invention relates to rice bran extracts that increase glucose uptake into cells that are useful for treating hypoglycemia, diabetes, metabolism, and obesity.

### BACKGROUND OF THE INVENTION

Type 2 Diabetes is characterized by disregulation of carbohydrate metabolism resulting in abnormally high level of sugar in blood (hyperglycemia). The characteristic symptoms, which severity increases with that abnormality, include (1) excessive urine production (polyuria) caused by sugar, resulting compensatory thirst and increased fluid intake (polydipsia); (2) blurred vision caused by sugar effects on the eye's optics; (3) unexplained weight loss; and, (4) lethargy. Type 1 diabetes, in which insulin is not produced or secreted by the pancreas, is usually due to autoimmune destruction of the pancreatic beta cells and is treatable only with injected insulin (K. I. Rother, 2007. Diabetes treatment—Bridging the divide. N. Eng. J. Med., 356:1499-1501). Type 2 diabetes is characterized by insulin resistance in target tissues and may be managed with a combination of dietary treatments, pharmaceuticals, and/or insulin supplementation (K. I. Rother, 2007. Diabetes treatment—Bridging the divide. N. Eng. J. Med., 356:1499-1501). As the disease progresses, there is a need for increasingly high levels of insulin and at some point the β-cells can no longer meet the demand. Gestational diabetes, often called preclampsia, involves insulin resistance (similar to type 2) caused by hormones of pregnancy in genetically predisposed women.

Diabetes can cause many complications. Acute complications like hypoglycemia, ketoacidosis, or nonketotic hyperosmolar coma may occur if the disease is not adequately controlled. Serious long-term complications include cardiovascular disease, chronic renal failure, retinal damage which can lead to blindness, nerve damage, and microvascular damage which may lead to poor healing (D. M. Nathan, 1993. Long-term complications of diabetes mellitus. N. Eng. J. Med., 328:1676-1685). Poor healing of wounds, particularly of the feet, can lead to gangrene, which may require amputation. Adequate treatment of diabetes, as well as increased emphasis on blood pressure control, can improve the risk profile of the aforementioned complications.

Diet has shown to play a definitive role in the onset of type 2 diabetes and the high refined sugar and high fat content of western diets are likely to be responsible for the increase in incidence of diabetes in the United States (J. S. Carter, J. A. Pugh and A. Monterrosa, 1996. Noninsulin-dependent diabetes mellitus in minorities in the United states. Ann. Intern. Med., 125:221-232). The recommended use of plants in the treatment of diabetes dates back to ca. 1550 BCE (A. M. Gray and P. R. Flatt, 1997. Pancreatic and extra-pancreatic effects of the traditional anti-diabetic plant, Medicago sativa (lucerne). Brit. J. Nutr., 78:325-334). Drug treatments are not feasible for a majority of the world's population, as such, alternative methods need to be evaluated and developed.

Rice bran, in particular, has been reported to have a number of healthful benefits and uses (Z. Takakori, M. Zare, M. Iranparvare, et al., 2005. Effect of rice bran on blood glucose and serum lipid parameters in diabetes II patients. Internet. J. Nutr. Wellness, .2:1; G. S. Seetharamaiah and N. Chandrasekhara, 1989. Studies on hypocholsterolemic activity of rice bran oil. Arthersclerosis, 78:219-223). Studies in Asia and India have also shown a significant reduction in serum cholesterol and triglyceride levels within a month of incorporating rice bran oil into the diet (Z. Takakori, M. Zare, M. Iranparvare and Y. Mehrabi, 2005. Effect of rice bran on blood glucose and serum lipid parameters in diabetes II patients. Internet. J. Nutr. Wellness, 2:1).

Rice bran contains tocotrienols and phytosterols. Biological activity associated with tocotrienols includes decreasing serum cholesterol, decreasing cholesterol synthesis, and anti-tumor activity (A. A. Quershi, N. Quershi, J. J. K. Wright, et al., 1991. Lowering of serum cholesterol in hypercholsterolemic humans by tocotrienols (palmvitee). Am. J. Clin. Nutr., 53:1021S-1026S; M. N. Gould, J. D. Haag, W. S. Kennan, et al., 1991. A comparison of tocopherol and tocotrienol for the chemoprevention of chemically induced rat mammary tumors. Am. J. Clin. Nutr., 53:1068S-1070S). The phytosterols in rice bran, particularly the oryzanols, are associated with decreased plasma cholesterol, platelet aggregation, hepatic biosynthesis of cholesterol, and cholesterol absorption (K. B. Wheeler and K. A. Garleb, 1991. g-Oryzanol-plant sterol supplementation: Metabolic, endocrine, and physiologic effects. Internatl. J. Sport Nutr., 1:170-177; G. S. Seetharamaiah and N. Chandrasekhara, 1990. Effect of oryzanol on cholesterol absoprtion and biliary and fecal bile acids in rats. Indian J. Med. Res., 92:471-475).

Glucose uptake is the process by which glucose in the blood is transported into the cells through very specific and different transport mechanisms. Glucose uptake can occur through facilitated diffusion and secondary active transport. Facilitated diffusion is an passive process that requires glucose uptake transporters (GLUT), particularly GLUT1 and GLUT3 which are responsible for maintaining a basal rate of glucose uptake (G. K. Brown, 2000. Glucose transporters: Structure, function, and consequences of deficiency. J. Inher. Metab. Disorders, 23:237-246). GLUT4 transporters are insulin sensitive, found in muscle and adipose tissue and, therefore, are important for post-prandial uptake of excess glucose from the bloodstream. Secondary active transport typically occurs in the kidneys and indirectly requires the hydrolysis of ATP, therefore is energy dependent (L. Reuss, 2000. One-hundred years of inquiry: The mechanism of glucose absorption in the intestine. Ann. Rev. Physiol., 62:939-946). There are two types of secondary active transporters, SGLT1 and SGLT2, found within the kidneys. SGLT1 has a high affinity but low capacity for glucose, whereas the opposite is true (low affinity, high capacity) for SGLT2 (T. Asano, M. Anai, H. Sakoda, et al., 2004. SGLT as a therapeutic target. Drugs Future, 29:461). The two SGLT transporters work together to ensure that as much glucose as possible is sent back into the bloodstream, and that only negligible amounts of glucose are excreted in the urine.

Impaired insulin-mediated glucose uptake is fundamental to the pathogenesis of type 2 diabetes thought the relationships are complex (R. A. DeFronzo, 1988. The triumvirate: beta-cell, muscle, liver. A collision responsible for NIDDM. Diabetes 37:667-687; A. Bsau, R Basu, P Shah, A Valla, C. M. Johnson, K. S. Nair, M. D. Jensen, W. F. Schwenk, and R. A. Rizza, 2000. Effects of type 2 diabetes on the ability of insulin and glucose to regulate splanchmic and muscle glucose metabolism. Evidence for a defect in hepatic glucokinase activity. Diabetes, 49:272-283; A. R. Cherrington, 1999. Control of glucose uptake and release by liver in vivo. Diabetes, 48:1198-1214; P. Iozzo, K. Hallstein, V. Oikonen, K. A. Virtanen, J. Kemppainen, O. Solin, E. Ferrannini, J. Knuuti and P. Nuutila, 2003. Insulin-mediated hepatic glucose uptake is impaired in type 2 diabetes: evidence for a relationship with glycemic control. J. Clin. Endrocrin. Metab., 88:2055-2060; P-H, Ducluzeau, L. M. Fletcher, H. Vidal, M. Laville, and J. M. Tavare, 2002. Molecular mechanisms of insulin-stimulated glucose uptake in adipocytes. Diabetes Metab. 28:85-92). In addition, a close relationship between enhanced glucose uptake—caloric excess—and increased synthesis and storage of lipids has linked type 2 diabetes with obesity (D. A. McClain, M. Hazel, G. Parker, and R. C. Cooksey, 2005. Adipocytes with increased hexoamine flux exhibit insulin resistance, increased glucose uptake, and increased synthesis and storage of lipid. Am. J. Physiol. Endrocrinol. Metab., 288:E973-E979; J. V. Nielsen and E. A. Joensson, 2008.Low-carbohydrate diet in type 2 diabetes: stable improvement of bodyweight and glycemic control during 44 months follow-up. Nutr. Metab., 5:1-6; S. Z. Yanovski and J. A. Yanovski, 2002. Obesity. New Eng. J. Med., 346:591-602). McClain et al. (2005) in particular, showed that insulin-stimulated glucose uptake was concomitant with a 41% increase in GLUT4 mRNA and a 206% increase in lipid synthesis, supporting the close relationships between enhanced glucose uptake and fat synthesis.

The role of the PPAR (peroxisome proliferator-activated receptor) nuclear receptor family, and particularly PPAR-γ, in control of glucose uptake in adipocytes is well established (T. M. Wilson, J. E. Cobb, D. J. Cowan, et al., 1996. The structure-activity relationship between peroxisome proliferator-activated receptor-γ agonism and the antihyperglycemic activity of thiazolidinediones. J. Med Chem., 39:665-668; R. Mukherjee, P. A. Hoener, L. Jow, J. Bilakovics, K. Klausing, D. E. Mais, A. Faulkner, G. E. Croston, and J. R. Paterniti, Jr., 2000. A selective perioxisome proliferator-activated receptor-γ (PPARγ) modular blocks adipocytes differentiation but stimulates glucose uptake in 3T3-L1 adipocytes. Molec. Endocrin., 14:1425-1433; C. Nugent, J. B. Prins, J. P. Whitehead, D. Savage, J. W. Wentworth, V. K. Chatterjee, and S. O'Rahilly, 2001. Potentiation of glucose uptake in 3T3-L1 adipocites by PPARγ agonists is maintained in cells expressing a PPARγ dominant-negative mutant: Evidence for selectivity in the downstream responses to PPARγ activation. Molec. Endrocrin., 15:1729-1738). PPARγ activation is critical to adipogenesis and therefore antagonist of this receptor could be useful in obesity, but importantly could prevent insulin-resistance and increase glucose uptake.

Characteristic of insulin resistance in type 2 diabetes is the generation of GLUT4 transporter in β-cell plasma membranes (D. E. James and R. C. Piper, 1994. Insulin resistance, diabetes, and the insulin regulated trafficking of GLUT4. J. Cell Biol., 126:1123-1126). Other studies have shown that in heterozygous GLUT4 knock-out mice that the insulin signally pathways can compensate for reduced levels of GLUT4 expression and function, but that cellular GLUT4 content is the rate-limiting factor in mediating maximal insulin-stimulated glucose uptake in adipocytes (L. I. Jing, K. L. Houseknecht, A. E. Stenbit, E. B. Katz, and M. J. Charron, 2000. Reduced glucose uptake precedes insulin signaling defects in adipocytes from heterozygous GLUT4 knockout mice. FASEB J., 14:1117-1125). It is currently thought that reduced insulin-stimulated glucose uptake is a result of abnormalities in insulin signaling pathways, including PI 3-kinase-dependent pathways, that control GLUT4 translocation to the plasma membrane (P-H, Ducluzeau, L. M. Fletcher, H. Vidal, M. Laville, and J. M. Tavare, 2002. Molecular mechanisms of insulin-stimulated glucose uptake in adipocytes. Diabetes Metab., 28:85-92). The cytoskeleton plays a critical role in vesicle trafficking related to control of glucose uptake via GLUT4 as disruption of these structures inhibits insulin-stimulated glucose uptake (A. Guilherme, M. Emoto, J. M. Buxton, S. Bose, R. Sabini, W. E. Theurkauf, J. Leszyk and M. P. Czech, 2000. Perinuclear localization and insulin-responsiveness of GLUT4 requires cytoskeletal integrity in 3T3-L1 adipocyctes. J. Biol. Chem., 275:38151-38159; A. L. Olsen, A. R. Trumbly, and G. V. Gibson, 2001. Insulin-mediated GLUT4 translocation is dependent on the microtubule network J. Biol. Chem., 276:10706-10714; P-H, Ducluzeau, L. M. Fletcher, H. Vidal, M. Laville, and J. M. Tavare, 2002. Molecular mechanisms of insulin-stimulated glucose uptake in adipocytes. Diabetes Metab., 28:85-92). Recycling endosomes become GLUT4 storage vesicles which are subsequently mobilized by the cytoskeleton for transport, docking to and fusion with the plasma membrane (K. J. Rodnick, J. W. Slot, D. R. Studelska, D. E. Hanpeter, L. J., L. J. Robinson, H. J. Geuze and D. E. James, 1992. Immunoctrochemical and biochemical studies of GLUT4 in rat skeletal muscle. J. Biol. Chem., 267:6278-6285). Insulin entry into adipocytes via the Insulin Receptor modulates the trafficking of the GLUT4 vesicles to the plasma membrane.

Fatty Acid Binding Proteins (FABP) are a multi-gene super family of lipid binding proteins (LBPs) involved in the transport of fatty acids and other lipids in various regions of the body (A. Chmurzynska, 2006. The multigene family of fatty acid-binding proteins (FABPs): function, structure and polymorphism. J. Appl. Genet. 47: 39-48). Regulation of fatty acid transport by FABP4 is important throughout the body as fatty acids are important sources of energy, building blocks for other molecules, and signaling molecules (E. Z. Amri, G. Ailhaud, et al., 1994. Fatty acids as signal transducing molecules: involvement in the differentiation of preadipose to adipose cells. J. Lipid Res., 35: 930-937; D. A., Bernlohr, N. R. Coe, et al., 1997. Regulation of gene expression in adipose cells by polyunsaturated fatty acids. Adv. Exp. Med. Biol. 422: 145-56; J. A. Hamilton, 1998. Fatty acid transport: difficult or easy? J. Lipid Res. 39:467-81).

FABPs can be subdivided into two major groups, the cytoplasmic FABPs (FABP_{c}) and plasma membrane FABPs (FABPₚₘ) (J. F. Glatz, and G. J. van der Vusse, 1996. Cellular fatty acid-binding proteins: their function and physiological significance. Prog. Lipid Res. 35:243-82). Currently, there are 9 types of FABP known, localized in various parts of the body, including adipocytes, the nervous system, muscle, liver and testes. This localization is important for function specific FABP. Since these proteins play a critical role in transport of specific fatty acids, modulation of the FABP proteins is a potential therapy for numerous conditions.

One of the most important FABPs in the body is adipocytes FABP (a.k.a. FABP4, aP2 or A-FABP). FABP4 is primarily found in adipocytes, but also in ciliary ganglion, appendix, skin, and in the placenta (C. A Baxa, R. S. Sha, et al., 1989. Human adipocyte lipid-binding protein: purification of the protein and cloning of its complementary DNA. Biochemistry 28:8683-8690); A. Chmurzynska, 2006. The multigene family of fatty acid-binding proteins (FABPs): function, structure and polymorphism. J. Appl. Genet., 47:39-48). Several studies have shown indications that FABP4 are important in several ailments. One study has shown that FABP4 is required for airway inflammation, indicating a potential role for FABP4 inhibition as an asthma treatment (Shum, B. O., C. R. Mackay, et al., 2006. The adipocyte fatty acid-binding protein aP2 is required in allergic airway inflammation. J. Clin. Invest. 116:2183-2192). Several studies have shown FABP4 playing a critical role in type 2 diabetes, atherosclerosis, and obesity (J. B. Boord, S. Fazio, et al., 2002. Cytoplasmic fatty acid-binding proteins: emerging roles in metabolism and atherosclerosis. Curr. Opin. Lipidol. 13:141-147; Makowski, L. and G. S. Hotamisligil, 2005. The role of fatty acid binding proteins in metabolic syndrome and atherosclerosis. Curr. Opin. Lipidol. 16:543-548; Erbay, E., H. Cao, et al., 2007. Adipocyte/macrophage fatty acid binding proteins in metabolic syndrome. Curr. Atheroscler. Rep. 9:222-229; M. Furuhashi, and G. S. Hotamisligil, 2008. Fatty acid-binding proteins: role in metabolic diseases and potential as drug targets. Nat Rev Drug Discov. 7:489-503). In particular, mice deficient in FABP4 have been shown to reduce hyperinsulinemia and insulin resistance (G. S. Hotamisligil, R. S. Johnson, et al., 1996. “Uncoupling of obesity from insulin resistance through a targeted mutation in aP2, the adipocyte fatty acid binding protein. Science 274: 1377-1379).

Using inhibitors of FABP4 for diabetes and atherosclerosis has been shown to be effective in mouse models (Furuhashi, M., G. Tuncman, et al., 2007. Treatment of diabetes and atherosclerosis by inhibiting fatty-acid-binding protein aP2. Nature 447:959-965). Future studies may elicit treatments for diabetes, atherosclerosis, asthma, some forms of inflammation and obesity by finding inhibitors of FABP4.

Several botanical-based bioactives have been shown to stimulate glucose uptake. Salidroside, a glycoside from Rhodiola rosea, stimulated glucose uptake in rat myoblast cells as well as insulin-mediated glucose uptake and this activity was mediated through AMP-activated protein kinase (H-B. Li, Y. Ge, X-X. Zheng and L. Zhang, 2008. Salidroside stimulated glucose uptake in skeletal muscle cells by activating AMP-activated protein kinase. Eur. J. Pharmacol., 588: 165-169). The isoquinoline alkaloid Berberine, which is found in certain Chinese Traditional Medicines derived from Coptidis rhizoma and Cortex phellodendri, has strong anti-hperglycemic effects (J. Yin, R. Hu, M. Chen, J. Tang, F. Li, Y. Yang, and J. Chen, 2002. Effects of berberine on glucose metabolism in vitro. Metab. Clin. Exper., 51:1439-1443; X. Bian, L. He, and G. Yang, 2006. Synthesis and antihyperglycemic evaluation of various protoberberine derivatives. Bioorgan. Med. Chem. Lett., 16:1380-1383; S. H. Kim, E-J. Shin, E-D. Kim, T. Bayarra, S. C. Frost and C-K. Hyun, 2007. Berine activates GLUT1-medeiated glucose uptake in 3T3-L1 adipocytes. Biol. Pharm. Bull., 30:2120-2125), and specifically activates GLUT1-mediated glucose transport in 3T3-L1 adipocyctes. A common flavonoid found in citrus, tomato and many berries, Naringenin, has been found to stimulate insulin-mediated glucose uptake (S. L. Lim, K. P. Soh, and U. R. Kuppusamy, 2008. Effects of naringenin on lipogensis, lipolysis and glucose uptake in Rat adipocytes primary culture: A nature antidiabetic agent. Internet. J. Altern. Med., 5:2), while Shikonin (5,8-dihydroxy-2-(1-hydroxy-4-methyl-pent-3-enyl)naphthalene-1,4-dione) a major component of Zicao (purple gromwell, the dried root of Lithospermum erythrorhizon) a Chinese herbal medicine, stimulates glucose uptake via an insulin-insensitive tyrosine kinase pathway (R. Kamei, Y. Kitagawa, M. Kadokura, F. Hattori, O. Hazeki, Y. Ebina, T. Nishihara, and S. Oikawa, 2002. Shinkonin stimulates glucose uptake in 3T2-L1 adipocytes via and insulin-independent tyrosine kinase pathway. Biochem. Biophys. Res. Commun., 292:642-651).

Cinnamon bark extracts have been shown to be active in glucose uptake stimulation and found to mitigate features of type 2 diabetes based on human clinical trials (A. Khan, M. Safdar, M. M. Khan, K. N. Khattak, and R. A. Anderson, 2003. Cinnamon improves glucose and lipids of people with type 2 diabetes, Diabetes Care, 26:3215-3218; E. J. Verspohl, K. Bauer, and E. Neddermann, 2005. Antidiabetic effect of Cinnamomum cassia and Cinnamomum zeylanicum in vivo and in vitro, Phytother. Res., 19:203-206; R. A. Anderson, J. H. Brantner, and M. M. Polansky, 1978. An improved assay for biologically active chromium, J. Agric. Food Chem., 26:1219-1221.

Other common botanical compounds like tannic acid stimulate glucose uptake via an insulin-dependent pathway (X. Liu, J-k. Kim, Y. Li, J. Li, F. Liu and X. Chen, 2005. Tannic acid stimulates glucose transport and inhibits adipocytes differentiation in 3T3-L1 cells. J. Nutr., 135:165-171), while palmitic acid enhances glucose-uptake via an insulin-dependent pathway that involves intracellular calcium mediation (J. Thode, H. A Pershadsingh, J. H. Ladenson, R. Hardy and J. M. McDonald, 1989. Palmitic acid stimulates glucose incorporation in the adipocyte by mechanisms likely involving intracellular calcium. J. Lipid Res., 30:1299-1305). Perrini et aL (S. Perrini, A. Natalicchio, L. Laviola, et al., 2004. Dehdroepiandrosterone stimulates glucose uptake in human and murine adipocytes by inducing GLUT1 and GLUT4 translocation to the plasma membrane. Diabetes, 53:41-52) have shown that DHEA (dehydroepiandrosterone) significantly stimulates glucose uptake and translocation of GLUT1 and GLUT4 translocator proteins to the plasma membrane via tyrosine phosphorylation of insulin receptor substrate (IRS-1) and IRS-2 and increases in intracellular calcium. In contrast, certain flavonoids like quercitin, myricetin and isoquercitin, which are very abundant in many fruit and vegetables, have been shown to be effective inhibitors of glucose uptake that is mediated via GLUT2 transporters, while inhibition of GLUT1 and GLUT4 has also been indicated (P. Strobel, C. Allard, T. Perez-Acle, T. Calderon, R. Adunate, and F. Leighton, 2005. Myricitin, quercetin, catechin-gallate inhibit glucose uptake in isolated rat adipocytes. Biochem. J., 386:471-4768; O. Kwon, P. Eck, S. Chen, C. P. Corpe, J-H. Lee, M. Kruhlak, and M. Levine, 2007. Inhibition of intestinal glucose transported GLUT2 by flavonoids. FASEB J., 21:366-377). More recently, it has been shown that quercetin and glucose pass through the GLUT1 transporter in the same manner and that quercetin binding blocks glucose transport based on docking studies (R. Cunningham, I. Afazal-Ahmed, and R. J. Naftalin, 2006. Docking studies show that D-glucose and quercetin slide through the transporter GLUT1. J. Bio. Chem., 281:5797-5803). It appears that quercitin is a competitive inhibitor of GLUT1. Inhibitors of glucose transport via GLUT1 and GLUT2 make have utility to address obesity and specific inhibitors of glucose transport in the small intestine (D. Cermak, S. Landgraf, and S. Wolffram, 2004. Quercitin glucides inhibit glucose uptake into brush-border-membrane vesicles of porcine jejunum. Brit. J. Nutr., 91:849-855). Fatty acids, particularly arachidonic acid, have been shown to stimulate glucose uptake through cycoloxygenase-independent mechanisms by increasing GLUT1 and GLUT4 activity in plasma membranes (J. B. P. Claire Nugent, P. Jonathan Whitehead, J. M. Wentworth, V. Krishna K. Chatterjee, and S. O'Rahilly, 2001. Arachidonic acid stimulates glucose uptake in 3T3-L1 adipocytes by increasing GLUT1 and GLUT4 levels at the plasma membrane. J. Biol. Chem., 278:9149-9157).

Disclosed below are optimized extracts from the stabilized bran of rice that enhance glucose uptake in human cells. The extracts show in vitro glucose uptake enhancing activity in the microgram per milliliter range (e.g., <1000 μg mL⁻¹). The extracts also possess FABP4 inhibition activity that promotes balanced fatty acid and carbohydrate metabolism key in diabetes and obesity. As such, the stabilized rice bran extracts are useful for treating hypoglycemia, diabetes, metabolic disorder, and obesity. In addition such extracts are safe, effective, and that can be provided as dietary supplements, added to multiple vitamins, and incorporated into foods to create functional foods.

### SUMMARY OF THE INVENTION

The present invention relates in part to a rice bran extract comprising at least one compound selected from the group consisting of 0.001 to 5% by weight of 2-methyl-butenoic acid, 0.001 to 5% by weight of 8-methyl-8-azabicyclo[3.2.1]octane-3,6-diol, 0.01 to 5% by weight of 4-isopropyl-1,2-benzenediol di-methyl ether, 0.005 to 5% by weight of glutamine N 5-isopropyl, 0.05 to 10% by weight of 6,10,14-trimethyl-5,9,13-pentadecatriene-2-one, 0.05 to 10% by weight of 11, 14 octadecadienal, 0.05 to 10% by weight of 9,11,13,15-octadecatetraenoic acid, 0.1 to 20% by weight of 7-hydroxy-14,14-dinor-8(17)-labden-13-one, 0.05 to 20% by weight of 9,12-octadecenoic acid, 0.05 to 20% by weight of 10-octadecenoic acid, 0.01 to 15% by weight of 16-hydroxy-9,12,14-octadecatrienoic acid, 0.05 to 15% by weight of 13-oxo-9-octadecenoic acid, 0.01 to 5% by weight of 4-oxooctadecenoic acid, 0.05 to 5% by weight of palmidrol, 0.005 to 5% by weight of fortimicin, 0.005 to 5% by weight of loeserinine, 0.01 to 5% by weight of 1,2-dihydroxy-5-heneicosen-4-one, 0.005 to 5% by weight of 2-amino-4-octadecene-1,3-diol, 0.01 to 5% by weight of 2-(aminomethyl)-2-propenoic acid N-hexadecanoyl methyl ester, 0.01 to 10% by weight of glycerol 1-alkanoates glycerol 1-octadecadienoate, 0.01 to 5% by weight of cyclobuxophylline O, 0.01 to 20% by weight of glycerol 1-alkanoates glycerol 1-octadecenoate, 0.01 to 5% by weight of buxandonine L, 0.005 to 5% by weight of 12-hydroxy-25-nor-17-scalarene-24-al, 0.005 to 5% by weight of coniodine A and 0.05 to 10% by weight of 24-nor-4(23),9(11)-fernidine.

Another aspect of the invention relates to a rice bran extract comprising at least one compound selected from the group consisting of 0.01 to 1% by weight of 2-methyl-butenoic acid, 0.01 to 2% by weight of 8-methyl-8-azabicyclo[3.2.1]octane-3,6-diol, 0.1 to 3% by weight of 4-isopropyl-1,2-benzenediol di-methyl ether, 0.01 to 1% by weight of glutamine N 5-isopropyl, 0.1 to 3% by weight of 6,10,14-trimethyl-5,9,13-pentadecatriene-2-one, 0.1 to 2% by weight of 11, 14 octadecadienal, 0.2 to 5% by weight of 9,11,13,15-octadecatetraenoic acid, 1 to 10% by weight of 7-hydroxy-14,14-dinor-8(17)-labden-13-one, 0.3 to 5% by weight of 9,12-octadecenoic acid, 0.2 to 5% by weight of 10-octadecenoic acid, 0.5 to 5% by weight of 16-hydroxy-9,12,14-octadecatrienoic acid, 0.5 to 5% by weight of 13-oxo-9-octadecenoic acid, 0.2 to 1% by weight of 4-oxooctadecenoic acid, 0.1 to 1% by weight of palmidrol, 0.01 to 0.5% by weight of fortimicin, 0.1 to 1% by weight of loeserinine, 0.1 to 1% by weight of 1,2-dihydroxy-5-heneicosen-4-one, 0.05 to 1% by weight of 2-amino-4-octadecene-1,3-diol, 0.1 to 1% by weight of 2-(aminomethyl)-2-propenoic acid N-hexadecanoyl methyl ester, 0.2 to 2% by weight of glycerol 1-alkanoates glycerol 1-octadecadienoate, 0.1 to 1% by weight of cyclobuxophylline 0, 0.1 to 2% by weight of glycerol 1-alkanoates glycerol 1-octadecenoate, 0.1 to 1% by weight of buxandonine L, 0.05 to 0.5% by weight of 12-hydroxy-25-nor-17-scalarene-24-al, 0.05 to 1% by weight of coniodine A and 0.2 to 2% by weight of 24-nor-4(23),9(11)-fernidine.

Still another aspect of the invention relates to a rice bran extract comprising at least one compound selected from the group consisting of 1 to 100 μg of 2-methyl-butenoic acid, 0.1 to 1000 μg of 8-methyl-8-azabicyclo[3.2.1]octane-3,6-diol, 10 to 2000 μg of 4-isopropyl-1,2-benzenediol di-methyl ether, 1 to 500 μg glutamine N 5-isopropyl, 100 to 2500 μg of 6,10,14-trimethyl-5,9,13-pentadecatriene-2-one, 100 to 2000 μg of 11, 14 octadecadienal, 100 to 2000 μg of 9,11,13,15-octadecatetraenoic acid, 500 to 15,000 μg of 7-hydroxy-14,14-dinor-8(17)-labden-13-one, 100 to 15,000 μg of 9,12-octadecenoic acid, 100 to 15,000 of 10-octadecenoic acid, 100 to 2500 μg of 16-hydroxy-9,12,14-octadecatrienoic acid, 100 to 5000 μg of 13-oxo-9-octadecenoic acid, 100 to 1500 μg of 4-oxooctadecenoic acid, 100 to 1500 μg of palmidrol, 5 to 200 of fortimicin, 20 to 1000 μg of loeserinine, 10 to 500 μg of 1, 2-dihydroxy-5-heneicosen-4-one, 10 to 500 μg of 2-amino-4-octadecene-1,3-diol, 10 to 500 μg of 2-(aminomethyl)-2-propenoic acid N-hexadecanoyl methyl ester, 100 to 2500 μg 1-alkanoates glycerol 1-octadecadienoate, 10 to 1000 μg cyclobuxophylline O, 100 to 3000 μg of glycerol 1-alkanoates glycerol 1-octadecenoate, 50 to 1000 μg of buxandonine L, 10 to 500 μg of 12-hydroxy-25-nor-17-scalarene-24-al, 10 to 500 μg of coniodine A, and 100 to 2000 of 24-nor-4(23),9(11)-fernidine, per 100 mg of extract.

Yet another aspect of the invention relates to a rice bran extract comprising at least one compound selected from the group consisting of 0.01 to 10% by weight of 4,5-dihydro-4-hydroxy-5-methyl-2-tetradecyl-2(3H)-furanone, 0.01 to 10% by weight of pregnane-2,3,6-triol, 0.01 to 10% by weight of 5-(8-heptadecenyl)dihydro-3-hydroxy-2(3H)-furanone, 0.01 to 10% by weight of 24-nor-4(23),9(11)-fernadine, 0.01 to 10% by weight of 24-nor-12-ursene, 0.01 to 10% by weight of 11,13(18)-oleanadiene, 0.01 to 5% by weight of 14-methyl-9,19-cycloergost-24(28)-en-3-ol, 0.01 to 10% by weight of montecristin, 0.01 to 10% by weight of 3-(3,4-dihydroxyphenyl)-2-propenoic acid triacontyl ester, 0.01 to 10% by weight of bombiprenone, and 0.001 to 10% by weight of glycerol 1,2-di-(9Z,12Z-octadecadienoate).

Another aspect of the invention relates to a rice bran extract comprising at least one compound selected from the group consisting of 0.1 to 2% by weight of 4,5-dihydro-4-hydroxy-5-methyl-2-tetradecyl-2(3H)-furanone, 0.1 to 2% by weight of pregnane-2,3,6-triol, 0.1 to 3% by weight of 5-(8-heptadecenyl)dihydro-3-hydroxy-2(3H)-furanone, 0.1 to 2% by weight of 24-nor-4(23),9(11)-fernadine, 0.5 to 5% by weight of 24-nor-12-ursene, 0.05 to 3% by weight of 11,13(18)-oleanadiene, 0.05 to 1% by weight of 14-methyl-9,19-cycloergost-24(28)-en-3-ol, 0.05 to 3% by weight of montecristin, 0.05 to 5% by weight of 3-(3,4-dihydroxyphenyl)-2-propenoic acid triacontyl ester, 0.01 to 10% by weight of bombiprenone, and 0.01 to 2% by weight of glycerol 1,2-di-(9Z,12Z-octadecadienoate).

Another aspect of the invention relates to a rice bran extract comprising at least one compound selected from the group consisting of 50 to 3000 μg of 4,5-dihydro-4-hydroxy-5-methyl-2-tetradecyl-2(3H)-furanone, 50 to 3000 μg of pregnane-2,3,6-triol, 50 to 3000 μg of 5-(8-heptadecenyl)dihydro-3-hydroxy-2(3H)-furanone, 50 to 2000 μg of 24-nor-4(23),9(11)-femadine, 10 to 5000 μg of 24-nor-12-ursene, 25 to 2500 μg of 11,13(18)-oleanadiene, 10 to 1000 μg of 14-methyl-9,19-cycloergost-24(28)-en-3-ol, 10 to 3000 μg of montecristin, 5 to 5000 μg of 3-(3,4-dihydroxyphenyl)-2-propenoic acid triacontyl ester, 5 to 5000 of bombiprenone, and 5 to 3000 μg of glycerol 1,2-di-(9Z, 12Z-octadecadienoate), per 100 mg of extract.

In some embodiments, the present invention relates to a rice bran extract, such as any of the aforementioned extracts, having a fraction comprising a Direct Analysis in Real Time (DART) mass spectrometry chromatogram of any of FIGS. 1 to 14.

In some embodiments, the rice bran extract has a glucose uptake stimulation greater than a glucose uptake stimulation of 200 nM insulin. In some embodiments, the glucose uptake stimulation of the extract is 0.5 to 5 times greater than the glucose uptake stimulation of 200 nM insulin. In some embodiments, the glucose uptake stimulation of the extract is 0.5 to 3.5 times greater than the glucose uptake stimulation of 200 nM insulin. In some embodiments, the glucose uptake stimulation of the extract is 0.7 to 3.1 times greater than the glucose uptake stimulation of 200 nM insulin. In other embodiments, the glucose uptake stimulation of the extract is more than 3 times greater than the glucose uptake stimulation of 200 nM insulin. In other embodiments, the glucose uptake stimulation of the extract is about 3 times greater than the glucose uptake stimulation of 200 nM insulin.

In another embodiment, the extract has a glucose uptake stimulation greater than a glucose uptake stimulation of control. In some embodiments, the extract glucose uptake stimulation is more than 1 times greater than the glucose uptake stimulation of control. In other embodiments, the extract glucose uptake stimulation is 1 to 10 times greater than the glucose uptake stimulation of control. In other embodiments, the extract glucose uptake stimulation is 2 to 7 times greater than the glucose uptake stimulation of control. In other embodiments, the extract glucose uptake stimulation is about 6 times greater than the glucose uptake stimulation of control.

In some embodiments, the extract has a glucose uptake stimulation of 100 to 3000 counts per minute (cpm). In other embodiments, the extract has a glucose uptake stimulation of 100 to 1000 cpm. In some embodiments, the concentration of the extract is 5 to 2000 μg/mL and the glucose uptake stimulation of 100 to 3000 cpm or 100 to 1000 cpm. In other embodiments, the concentration of extract is 10 to 1000 μg/mL. In other embodiments, the concentration of extract is 10, 50, 250 or 1000 μg/mL.

In some embodiments, the rice bran extract has an IC₅₀ value for FABP4 inhibition of less than 2000 μg/mL. In other embodiments, the IC₅₀ value for FABP4 inhibition is from 25 to 2000 μg/mL, from 25 to 1000 μg/mL, or from 25 to 500 μg/mL.

Another aspect of the invention relates to a rice bran extract prepared by a process comprising the following steps:
a) providing a stabilized rice bran feedstock, and
b) extracting the feedstock.
   In some embodiments, the extracting step is an aqueous ethanol extraction, while in other embodiments, the extracting step is supercritical carbon dioxide extraction.

Another aspect of the invention relates to a pharmaceutical composition comprising any of the aforementioned rice bran extracts. In some embodiments, the rice bran extract is formulated as a functional food, dietary supplement, powder or beverage.

Another aspect of the invention relates to a method of inhibiting glucose uptake comprising administering to a subject in need thereof an effective amount of any of the aforementioned rice bran extracts or pharmaceutical compositions.

Another aspect of the invention relates to a method if inhibiting FABP4 binding comprising administering to a subject in need thereof an effective amount of any of the aforementioned rice bran extracts or pharmaceutical compositions. In some embodiments, the subject has hyperglycemia. In other embodiments, the subject has diabetes. In other embodiments, the subject has type 1 diabetes, while in other embodiments, the subject has type 2 diabetes. In other embodiments, the subject has obesity and related metabolic disorders.

### BRIEF DESCRIPTION OF THE DRAWINGS

FIG. 1 depicts a DART TOF-MS spectrum of SRB Extract 1 obtained by extraction at room temperature with 80% (v/v) ethanol, with the X-axis showing the mass distribution (100-800 m/z [M+H+]) and the y-axis showing the relative abundances of each chemical species of the detected.

FIG. 2 depicts a DART TOF-MS spectrum of SRB Extract 2 obtained by extraction at 40° C. with distilled water, with the X-axis showing the mass distribution (100-800 m/z [M+H+]) and the y-axis showing the relative abundances of each chemical species of the detected.

FIG. 3 depicts a DART TOF-MS spectrum of SRB Extract 3 obtained by extraction at 40° C., with 20% (v/v) ethanol the X-axis showing the mass distribution (100-800 m/z [M+H+]) and the y-axis showing the relative abundances of each chemical species of the detected.

FIG. 4 depicts a DART TOF-MS spectrum of an SRB Extract 4 obtained by extraction at 40° C. with 40% (v/v) ethanol the X-axis showing the mass distribution (100-800 m/z [M+H+]) and the y-axis showing the relative abundances of each chemical species of the detected.

FIG. 5 depicts a DART TOF-MS spectrum of SRB Extract 5 obtained by extraction at 40° C. with 60% (v/v) ethanol the X-axis showing the mass distribution (100-800 m/z [M+H+]) and the y-axis showing the relative abundances of each chemical species of the detected.

FIG. 6 depicts a DART TOF-MS spectrum of SRB Extract 6 (extracted at 40° C., 80% [v/v] ethanol), with the X-axis showing the mass distribution (100-800 m/z [M+H+]) and the y-axis showing the relative abundances of each chemical species of the detected.

FIG. 7 depicts a DART TOF-MS spectrum of SRB Extract 7 obtained by extraction at 40° C. with 100% ethanol the X-axis showing the mass distribution (100-800 m/z [M+H+]) and the y-axis showing the relative abundances of each chemical species of the detected.

FIG. 8 depicts a DART TOF-MS spectrum of SRB Extract 8 obtained by extraction at 60° C. with 80% (v/v) ethanol the X-axis showing the mass distribution (100-800 m/z [M+H+]) and the y-axis showing the relative abundances of each chemical species of the detected.

FIG. 9 depicts a DART TOF-MS spectrum of SRB Extract 9 (obtained by SCCO2 extraction at 40° C., 300 bar), with the X-axis showing the mass distribution (100-800 m/z [M+H+]) and the y-axis showing the relative abundances of each chemical species of the detected.

FIG. 10 depicts a DART TOF-MS spectrum of SRB extract 10 obtained by SCCO2 extraction at 40° C., 500 bar, the X-axis showing the mass distribution (100-800 m/z [M+H+]) and the y-axis showing the relative abundances of each chemical species of the detected.

FIG. 11 depicts a DART TOF-MS spectrum of SRB extract 11 obtained by SCCO2 extraction at 60° C., 300 bar, the X-axis showing the mass distribution (100-800 m/z [M+H+]) and the y-axis showing the relative abundances of each chemical species of the detected.

FIG. 12 depicts a DART TOF-MS spectrum of SRB extract 12 obtained by SCCO2 extraction at 60° C., 500 bar, the X-axis showing the mass distribution (100-800 m/z [M+H+]) and the y-axis showing the relative abundances of each chemical species of the detected.

FIG. 13 depicts a DART TOF-MS spectrum of SRB extract 13 obtained by SCCO2 extraction at 80° C., 300 bar, the X-axis showing the mass distribution (100-800 m/z [M+H+]) and the y-axis showing the relative abundances of each chemical species of the detected.

FIG. 14 depicts a DART TOF-MS spectrum of SRB extract 14 obtained by SCCO2 extraction at 80° C., 500 bar, the X-axis showing the mass distribution (100-800 m/z [M+H+]) and the y-axis showing the relative abundances of each chemical species of the detected.

### DETAILED DESCRIPTION OF THE INVENTION

### Definitions

The term “Synergistic” is art recognized and refers to two or more components working together so that the total effect is greater than the sum of the components.

The term “Treating” is art-recognized and refers to curing as well as ameliorating at least one symptom of any condition or disorder.

As used herein, the term “Beta cells or β-cells” refers to a type of cell in the pancreas that makes and releases insulin, a hormone that controls the level of glucose in the blood.

As used herein, the term “Glucose uptake” refers to the process of glucose being taken into cells. The method of glucose uptake differs throughout tissues depending on two factors; the metabolic needs of the tissue and availability of glucose. The two ways in which glucose uptake can take place are facilitated diffusion (a passive process) and secondary active transport (an active process which indirectly requires the hydrolysis of ATP).

As used herein, the term “3T3-L1 cells” refers to a cell line derived from 3T3 cells that is used in biological research on adipose tissue. These cells have a fibroblast-like morphology, but, under appropriate conditions, the cells differentiate into an adipocyte-like phenotype. The 3T3-L1 cells of the adipocyte morphology increase the synthesis and accumulation of triglycerides and acquire the signet ring appearance of adipose cells. These cells are also sensitive to lipogenic and lipolytic hormones and drugs, including epinephrine, isoproterenol, and insulin.

As used herein, the term “GLUT” refers to glucose transporters and represent a family of membrane proteins found in many mammalian cells. GLUTs are integral membrane proteins which contain 12 membrane spanning helices with both the amino and carboxyl termini exposed on the cytoplasmic side of the plasma membrane. GLUT proteins transport glucose and related hexoses according to a model of alternate conformation, which predicts that the transporter exposes a single substrate binding site toward either the outside or the inside of the cell. Binding of glucose to one site provokes a conformational change associated with transport, and releases glucose to the other side of the membrane. The inner and outer glucose-binding sites are probably located in transmembrane segments 9, 10, 11 of the transporter. Also, the QLS motif located in the seventh transmembrane segment could be involved in the selection and affinity of transported substrate. GLUT1 is responsible for the low-level of basal glucose uptake required to sustain respiration in all cells and GLUT1 levels in cell membranes are increased by reduced glucose levels and decreased by increased glucose levels. GLUT4 is found in adipose tissues and striated muscle (skeletal muscle and cardiac muscle) and is the insulin-regulated glucose transporter responsible for insulin-regulated glucose storage.

As used herein, the term “FABP” refers to Fatty Acid Binding Proteins (FABP) are a multi-gene super family of lipid binding proteins (LBPs) involved in the transport of fatty acids and other lipids in various regions of the body. FABPs can be subdivided into two major groups, the cytoplasmic FABPs (FABP_{c}) and plasma membrane FABPs (FABPₚₘ). There are 9 types of FABP known, localized in various parts of the body, including adipocytes, the nervous system, muscle, liver and testes. The localization is important for function specific FABPs.

As used herein, the term “FABP4” refers to a specific Fatty Acid Binding Protein 4 which is a key mediator of intracellular transport and metabolism of fatty acids in adipose tissues. FABP4 binds fatty acids with high affinity and transports them to various cellular compartments. FABP4, when complexed with fatty acids, interacts with and modulates the activity of two important regulators of metabolism, hormone-sensitive lipase and peroxisome proliferator-activated receptor gamma (PPAR-γ). FABP4 plays a critical role in Type 2 diabetes.

As used her, the term “Cytochalasin” or “Cytochalasin B” refers to cell-permeable mycotoxins. Cytochalasin B inhibits cytoplasmic division by blocking the formation of contractile microfilaments. It inhibits cell movement and induces nuclear extrusion. Cytochalasin B shortens actin filaments by blocking monomer addition at the fast-growing end of polymers, and specifically inhibits glucose transport and platelet aggregation.

As used here, the term “IRS-1” refers to Insulin Receptor Substrate-1 plays a key role in transmitting signals from the insulin and insulin-like growth factor-1 (IGF-1) receptors to intracellular pathways PI3K/AKT and Erk MAP kinase pathways. IRS-1 plays important roles in metabolic and mitogenic (growth promoting) pathways. For example mice deficient in IRS-1 have diabetic phenotype.

As used here, the term “IR” or Insulin Receptor” is a transmembrane receptor that is activated by insulin. It belongs to the large class of tyrosine kinase receptors. Two alpha subunits and two beta subunits make up the insulin receptor. The beta subunits pass through the cellular membrane and are linked by disulfide bonds. The alpha and beta subunits are encoded by a single gene (INSR).

As used here, the term “AKT” refers to Protein Kinase B important in mammalian signally. It is required for the insulin-induced translocation of glucose transporter 4 (GLUT4) to the plasma membrane. Glycogen synthase kinase 3 (GSK-3) can be inhibited upon phosphorylation by AKT, which results in promotion of glycogen synthesis. GSK-3 is involved in Wnt signaling and AKT might be also implicated in the Wnt pathway in control of cellular metabolism.

As used here, the term “Zucker rat” refers to a genetic line of brown rats (Rattus norvegicus) laboratory rat strain known as a Zucker rat. These rats are bred to be genetically prone to diabetes, the same metabolic disorder found among humans.

Extracts

The present invention relates in part to stabilized rice (SRB) extracts comprising certain compounds. In some embodiments, the rice bran extract comprises at least one compound selected from the group consisting of 0.001 to 5% by weight of 2-methyl-butenoic acid, 0.001 to 5% by weight of 8-methyl-8-azabicyclo[3.2.1]octane-3,6-diol, 0.01 to 5% by weight of 4-isopropyl-1,2-benzenediol di-methyl ether, 0.005 to 5% by weight of glutamine N 5-isopropyl, 0.05 to 10% by weight of 6,10,14-trimethyl-5,9,13-pentadecatriene-2-one, 0.05 to 10% by weight of 11, 14 octadecadienal, 0.05 to 10% by weight of 9,11,13,15-octadecatetraenoic acid, 0.1 to 20% by weight of 7-hydroxy-14,14-dinor-8(17)-labden-13-one, 0.05 to 20% by weight of 9,12-octadecenoic acid, 0.05 to 20% by weight of 10-octadecenoic acid, 0.01 to 15% by weight of 16-hydroxy-9,12,14-octadecatrienoic acid, 0.05 to 15% by weight of 13-oxo-9-octadecenoic acid, 0.01 to 5% by weight of 4-oxooctadecenoic acid, 0.05 to 5% by weight of palmidrol, 0.005 to 5% by weight of fortimicin, 0.005 to 5% by weight of loeserinine, 0.01 to 5% by weight of 1,2-dihydroxy-5-heneicosen-4-one, 0.005 to 5% by weight of 2-amino-4-octadecene-1,3-diol, 0.01 to 5% by weight of 2-(aminomethyl)-2-propenoic acid N-hexadecanoyl methyl ester, 0.01 to 10% by weight of glycerol 1-alkanoates glycerol 1-octadecadienoate, 0.01 to 5% by weight of cyclobuxophylline 0, 0.01 to 20% by weight of glycerol 1-alkanoates glycerol 1-octadecenoate, 0.01 to 5% by weight of buxandonine L, 0.005 to 5% by weight of 12-hydroxy-25-nor-17-scalarene-24-al, 0.005 to 5% by weight of coniodine A and 0.05 to 10% by weight of 24-nor-4(23),9(11)-fernidine. The extract may comprise one, two, or more of the aforementioned compounds, or the extract may contain all of the aforementioned compounds. In certain embodiments, the extract comprises all of the aforementioned compounds.

In some embodiments, the rice bran extract comprises at least one compound selected from the group consisting of 0.01 to 1% by weight of 2-methyl-butenoic acid, 0.01 to 2% by weight of 8-methyl-8-azabicyclo[3.2.1]octane-3,6-diol, 0.1 to 3% by weight of 4-isopropyl-1,2-benzenediol di-methyl ether, 0.01 to 1% by weight of glutamine N 5-isopropyl, 0.1 to 3% by weight of 6,10,14-trimethyl-5,9,13-pentadecatriene-2-one, 0.1 to 2% by weight of 11,14-octadecadienal, 0.2 to 5% by weight of 9,11,13,15-octadecatetraenoic acid, 1 to 10% by weight of 7-hydroxy-14,14-dinor-8(17)-labden-13-one, 0.3 to 5% by weight of 9,12-octadecenoic acid, 0.2 to 5% by weight of 10-octadecenoic acid, 0.5 to 5% by weight of 16-hydroxy-9,12,14-octadecatrienoic acid, 0.5 to 5% by weight of 13-oxo-9-octadecenoic acid, 0.2 to 1% by weight of 4-oxooctadecenoic acid, 0.1 to 1% by weight of palmidrol, 0.01 to 0.5% by weight of fortimicin, 0.1 to 1% by weight of loeserinine, 0.1 to 1% by weight of 1,2-dihydroxy-5-heneicosen-4-one, 0.05 to 1% by weight of 2-amino-4-octadecene-1,3-diol, 0.1 to 1% by weight of 2-(aminomethyl)-2-propenoic acid N-hexadecanoyl methyl ester, 0.2 to 2% by weight of glycerol 1-alkanoates glycerol 1-octadecadienoate, 0.1 to 1% by weight of cyclobuxophylline O, 0.1 to 2% by weight of glycerol 1-alkanoates glycerol 1-octadecenoate, 0.1 to 1% by weight of buxandonine L, 0.05 to 0.5% by weight of 12-hydroxy-25-nor-17-scalarene-24-al, 0.05 to 1% by weight of coniodine A and 0.2 to 2% by weight of 24-nor-4(23),9(11)-fernidine.

Still another aspect of the invention relates to a rice bran extract comprising at least one compound selected from the group consisting of 1 to 100 μg of 2-methyl-butenoic acid, 0.1 to 1000 μg of 8-methyl-8-azabicyclo[3.2.1]octane-3,6-diol, 10 to 2000 μg of 4-isopropyl-1,2-benzenediol di-methyl ether, 1 to 500 μg glutamine N 5-isopropyl, 100 to 2500 μg of 6,10,14-trimethyl-5,9,13-pentadecatriene-2-one, 100 to 2000 μg of 11, 14 octadecadienal, 100 to 2000 μg of 9,11,13,15-octadecatetraenoic acid, 500 to 15,000 μg of 7-hydroxy-14,14-dinor-8(17)-labden-13-one, 100 to 15,000 μg of 9,12-octadecenoic acid, 100 to 15,000 of 10-octadecenoic acid, 100 to 2500 μg of 16-hydroxy-9,12,14-octadecatrienoic acid, 100 to 5000 μg of 13-oxo-9-octadecenoic acid, 100 to 1500 μg of 4-oxooctadecenoic acid, 100 to 1500 μg of palmidrol, 5 to 200 of fortimicin, 20 to 1000 μg of loeserinine, 10 to 500 μg of 1, 2-dihydroxy-5-heneicosen-4-one, 10 to 500 μg of 2-amino-4-octadecene-1,3-diol, 10 to 500 μg of 2-(aminomethyl)-2-propenoic acid N-hexadecanoyl methyl ester, 100 to 2500 μg 1-alkanoates glycerol 1-octadecadienoate, 10 to 1000 μg cyclobuxophylline O, 100 to 3000 μg of glycerol 1-alkanoates glycerol 1-octadecenoate, 50 to 1000 μg of buxandonine L, 10 to 500 μg of 12-hydroxy-25-nor-17-scalarene-24-al, 10 to 500 μg of coniodine A, and 100 to 2000 of 24-nor-4(23),9(11)-fernidine, per 100 mg of extract.

In another embodiment, the rice bran extract comprises at least one compound selected from the group consisting of 25 to 75 μg of 2-methyl-butenoic acid, 300 to 500 μg of 8-methyl-8-azabicyclo[3.2.1]octane-3,6-diol, 750 to 100 μg of 4-isopropyl-1,2-benzenediol di-methyl ether, 100 to 250 μg glutamine N 5-isopropyl, 500 to 2000 μg of 6,10,14-trimethyl-5,9,13-pentadecatriene-2-one, 250 to 750 μg of 11, 14 octadecadienal, 1000 to 1500 μg of 9,11,13,15-octadecatetraenoic acid, 5000 to 10,000 μg of 7-hydroxy-14,14-dinor-8(17)-labden-13-one, 5000 to 10,000 μg of 9,12-octadecenoic acid, 200 to 1000 of 10-octadecenoic acid, 1000 to 2000 μg of 16-hydroxy-9,12,14-octadecatrienoic acid, 500 to 3000 μg of 13-oxo-9-octadecenoic acid, 200 to 800 μg of 4-oxooctadecenoic acid, 200 to 800 μg of palmidrol, 10 to 200 μg of fortimicin, 50 to 500 μg of loesenerine, 50 to 500 μg of 1,2-dihydroxy-5-heneicosen-4-one, 100 to 500 μg of 2-amino-4-octadecene-1,3-diol, 100 to 500 μg of 2-(aminomethyl)-2-propenoic acid N-hexadecanoyl methyl ester, 200 to 1000 μg 1-alkanoates glycerol 1-octadecadienoate, 100 to 1000 μg cyclobuxophylline O, 200 to 1000 μg of glycerol 1-alkanoates glycerol 1-octadecenoate, 200 to 1000 μg of buxandonine L, 10 to 500 μg of 12-hydroxy-25-nor-17-scalarene-24-al, 100 to 500 μg of coniodine A, and 500 to 1500 of 24-nor-4(23),9(11)-fernidine, per 100 mg of extract.

In some embodiments, the rice bran extract comprises about 5, 10, 20, 30, 40, 50, 60, 70, 80, 90 or 100 μg of 2-methyl-butenoic acid per 100 mg of the extract.

In some embodiments, the rice bran extract comprises about 10, 20, 30, 40, 50, 60, 70, 80, 90, 100, 150, 200, 250, 300, 350, 400, or 450 μg of 8-methyl-8-azabicyclo[3.2.1]octane-3,6-diol per 100 mg of extract.

In some embodiments, the rice bran extract comprises about 100, 200, 300, 400, 500, 600, 700, 800, 900 or 100 μg of 4-isopropyl-1,2-benzenediol di-methyl ether per 100 mg extract.

In some embodiments, the rice bran extract comprises about 10, 20, 30, 40, 50, 60, 70, 80, 90, 100, 110, 120, 130, 140, 150, 160, 170, 180, 190, or 200 μg of glutamine N 5-isopropyl per 100 mg of extract.

In some embodiments, the rice bran extract comprises about 200, 300, 400, 500, 600, 700, 800, 900, 1000, 1100, 1200, 1300, 1400, 1500, 1600, 1700, 1800, 1900, or 2000 μg of 6,10,14-trimethyl-5,9,13-pentadecatriene-2-one per 100 mg of extract.

In some embodiments, the rice bran extract comprises about 200, 300, 400, 500, 600, 700, 800, 900, 1000, 1100, 1200, 1300, 1400, or 1500 μg of 11, 14 octadecadienal per 100 mg of extract.

In some embodiments, the rice bran extract comprises about 200, 300, 400, 500, 600, 700, 800, 900, 1000, 1100, 1200, 1300, 1400 or 1500 μg of 9,11,13,15-octadecatetraenoic acid per 100 mg of extract.

In some embodiments, the rice bran extract comprises about 1000, 1500, 2000, 2500, 3000, 3500, 4000, 4500, 5000, 5500, 6000, 6500, 7000, 7500, 8000, 8500, 9000, 9500, or 10000 to 15,000 μg of 7-hydroxy-14,14-dinor-8(17)-labden-13-one per 100 mg of extract.

In some embodiments, the rice bran extract comprises about 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, or 10000 μg of 9,12-octadecenoic acid per 100 mg of extract.

In some embodiments, the rice bran extract comprises about 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, or 10000 μg to 15,000 of 10-octadecenoic acid per 100 mg of extract.

In some embodiments, the rice bran extract comprises about 500, 600, 700, 800, 900, 1000, 1100, 1200, 1300, 1400, 15000, 1600, 1700, 1800, 1900, or 2000 μg of 16-hydroxy-9,12,14-octadecatrienoic acid per 100 mg of extract.

In some embodiments, the rice bran extract comprises about 500, 600, 700, 800, 900, 1000, 1500, 2000, 2500, 3000, 3500, 4000, 4500, or 5000 μg of 13-oxo-9-octadecenoic acid per 100 mg of extract.

In some embodiments, the rice bran extract comprises about 100, 200, 300, 400, 500, 600, 700, 800, 900, or 1000 μg of 4-oxooctadecenoic acid per 100 mg of extract.

In some embodiments, the rice bran extract comprises about 100, 200, 300, 400, 500, 600, 700, 800, 900, or 1000 μg of palmidrol per 100 mg of extract.

In some embodiments, the rice bran extract comprises about 10, 20, 30, 40, 50, 60, 70, 80 90 or 100 μg of fortimicin per 100 mg of extract.

In some embodiments, the rice bran extract comprises about 100, 150, 200, 250, 300, 350, 400, 450, or 500 μg of loesenerine per 100 mg of extract.

In some embodiments, the rice bran extract comprises about 100, 110, 120, 130, 140, 150, 160, 170, 180, 190, 200, 210, 220, 230, 240, 250, 260, 270, 280, 290, or 300 μg of 1,2-dihydroxy-5-heneicosen-4-one per 100 mg of extract.

In some embodiments, the rice bran extract comprises about 50, 100, 150, 200, 250, 300, 250, 400, 450, or 500 μg of 2-amino-4-octadecene-1,3-diol per 100 mg of extract.

In some embodiments, the rice bran extract comprises about 100, 150, 200, 250, 300, 250, 400, 450, or 500 μg of 2-(aminomethyl)-2-propenoic acid N-hexadecanoyl methyl ester per 100 mg of extract.

In some embodiments, the rice bran extract comprises about 200, 300, 400, 500, 600, 700, 800, 900, 1000, 1100, 1200, 1300, 1400, 1500, 1600, 1700, 1800, 1900 or 2000 μg 1-alkanoates glycerol 1-octadecadienoate per 100 mg of extract.

In some embodiments, the rice bran extract comprises about 100, 150, 200, 250, 300, 350, 400, 450, or 500 μg cyclobuxophylline O per 100 mg of extract.

In some embodiments, the rice bran extract comprises about 200, 300, 400, 500, 600, 700, 800, 900, 1000, 1500, 2000, or 2500 μg of glycerol 1-alkanoates glycerol 1-octadecenoate per 100 mg of extract.

In some embodiments, the rice bran extract comprises about 100, 150, 200, 250, 300, 350, 400, 450, 500, 550, 600, 650, 700 or 750 μg of buxandonine L per 100 mg of extract.

In some embodiments, the rice bran extract comprises about 50, 60, 70, 80, 90, 100, 150, 200, 250, 300, 250, 400, or 500 μg of 12-hydroxy-25-nor-17-scalarene-24-al per 100 mg of extract.

In some embodiments, the rice bran extract comprises about 50, 60, 70, 80, 90, 100, 150, 200, 250, 300, 250, 400, or 500 μg of coniodine A per 100 mg of extract.

In some embodiments, the rice bran extract comprises about 200, 300, 400, 500, 600, 700, 800, 900, 1000, 1100, 1200, 1300, 1400, or 1500 μg of 24-nor-4(23),9(11)-fernidine per 100 mg of extract.

Yet another aspect of the invention relates to a rice bran extract comprising at least one compound selected from the group consisting of 0.01 to 10% by weight of 4,5-dihydro-4-hydroxy-5-methyl-2-tetradecyl-2(3H)-furanone, 0.01 to 10% by weight of pregnane-2,3,6-triol, 0.01 to 10% by weight of 5-(8-heptadecenyl)dihydro-3-hydroxy-2(3H)-furanone, 0.01 to 10% by weight of 24-nor-4(23),9(11)-fernadine, 0.01 to 10% by weight of 24-nor-12-ursene, 0.01 to 10% by weight of 11,13(18)-oleanadiene, 0.01 to 5% by weight of 14-methyl-9,19-cycloergost-24(28)-en-3-ol, 0.01 to 10% by weight of montecristin, 0.01 to 10% by weight of 3-(3,4-dihydroxyphenyl)-2-propenoic acid triacontyl ester, 0.01 to 10% by weight of bombiprenone, and 0.001 to 10% by weight of glycerol 1,2-di-(9Z,12Z-octadecadienoate). The extract may comprise one, two or more of the aforementioned compounds, or the extract may comprise all of the aforementioned compounds.

In some embodiments, the rice bran extract comprises at least one compound selected from the group consisting of 0.1 to 2% by weight of 4,5-dihydro-4-hydroxy-5-methyl-2-tetradecyl-2(3H)-furanone, 0.1 to 2% by weight of pregnane-2,3,6-triol, 0.1 to 3% by weight of 5-(8-heptadecenyl)dihydro-3-hydroxy-2(3H)-furanone, 0.1 to 2% by weight of 24-nor-4(23),9(11)-fernadine, 0.5 to 5% by weight of 24-nor-12-ursene, 0.05 to 3% by weight of 11,13(18)-oleanadiene, 0.05 to 1% by weight of 14-methyl-9,19-cycloergost-24(28)-en-3-ol, 0.05 to 3% by weight of montecristin, 0.05 to 5% by weight of 3-(3,4-dihydroxyphenyl)-2-propenoic acid triacontyl ester, 0.01 to 10% by weight of bombiprenone, and 0.01 to 2% by weight of glycerol 1,2-di-(9Z,12Z-octadecadienoate).

In some embodiments, the rice bran extract comprises at least one compound selected from the group consisting of 50 to 3000 μg of 4,5-dihydro-4-hydroxy-5-methyl-2-tetradecyl-2(3H)-furanone, 50 to 3000 μg of pregnane-2,3,6-triol, 50 to 3000 μg of 5-(8-heptadecenyl)dihydro-3-hydroxy-2(3H)-furanone, 50 to 2000 μg of 24-nor-4(23),9(11)-femadine, 10 to 5000 μg of 24-nor-12-ursene, 25 to 2500 μg of 11,13(18)-oleanadiene, 10 to 1000 μg of 14-methyl-9,19-cycloergost-24(28)-en-3-ol, 10 to 3000 μg of montecristin, 5 to 5000 μg of 3-(3,4-dihydroxyphenyl)-2-propenoic acid triacontyl ester, 5 to 5000 of bombiprenone, and 5 to 3000 μg of glycerol 1,2-di-(9Z,12Z-octadecadienoate), per 100 mg of extract.

In some embodiments, the rice bran extract comprises at least one compound selected from the group consisting of 100 to 1500 μg of 4,5-dihydro-4-hydroxy-5-methyl-2-tetradecyl-2(3H)-furanone, 100 to 1500 μg of pregnane-2,3,6-triol, 100 to 2500 μg of 5-(8-heptadecenyl)dihydro-3-hydroxy-2(3H)-furanone, 100 to 1500 μg of 24-nor-4(23),9(11)-fernadine, 50 to 1000 μg of 24-nor-12-ursene, 100 to 2000 μg of 1,13(18)-oleanadiene, 50 to 1000 μg of 14-methyl-9,19-cycloergost-24(28)-en-3-ol, 50 to 2500 μg 5 of montecristin, 10 to 1500 μg of 3-(3,4-dihydroxyphenyl)-2-propenoic acid triacontyl ester, 10 to 2500 of bombiprenone, and 10 to 2000 μg of glycerol 1,2-di-(9Z,12Z-octadecadienoate), per 100 mg of extract.

In some embodiments, the rice bran extract comprises about 100, 200, 300, 400, 500, 600, 700, 800, 900, 1000, 1100, 1200, 1300, 1400 or 1500 μg of 4,5-dihydro-4-hydroxy-5-methyl-2-tetradecyl-2(3H)-furanone per 100 mg of extract.

In some embodiments, the rice bran extract comprises about 100, 200, 300, 400, 500, 600, 700, 800, 900, 1000, 1100, 1200, 1300, 1400 or 1500 μg of pregnane-2,3,6-triol per 100 mg of extract.

In some embodiments, the rice bran extract comprises about 100, 200, 300, 400, 500,600,700,800,900, 1000, 1100, 1200, 1300, 1400, 1500, 1600, 1700, 1800, 1900, 2000, 2100, 2200, 2300, 2400, or 2500 μg of 5-(8-heptadecenyl)dihydro-3-hydroxy-2(3H)-furanone per 100 mg of extract.

In some embodiments, the rice bran extract comprises about 100, 200, 300, 400, 500, 600, 700, 800, 900, 1000, 1100, 1200, 1300, 1400, or 1500 μg of 24-nor-4(23),9(11)-femadine per 100 mg of extract.

In some embodiments, the rice bran extract comprises about 10, 20, 30, 40, 50, 60, 70, 80, 90, 100,200,300,400, 500, 600, 700, 800, 900, 1000, 1100, 1200, 1300, 1400, 1500, 200, 2500, or 3000 μg of 24-nor-12-ursene per 100 mg of extract.

In some embodiments, the rice bran extract comprises about 100, 200, 300, 400, 500,600,700,800,900, 1000, 1100, 1200, 1300, 1400, 1500, 1600, 1700, 1800, 1900, or 2000 μg of 11,13(18)-oleanadien per 100 mg of extract.

In some embodiments, the rice bran extract comprises about 100, 200, 300, 400, 500, 600, 700, 800, 900, or 1000 μg of 14-methyl-9,19-cycloergost-24(28)-en-3-ol per 100 mg of extract.

In some embodiments, the rice bran extract comprises about 100, 200, 300, 400, 500,600,700,800,900, 1000, 1100, 1200, 1300, 1400, 1500, 1600, 1700, 1800, 1900, 2000, 2100, 2200, 2300, 2400, 2500 μg of montecristin per 100 mg of extract.

In some embodiments, the rice bran extract comprises about 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500 μg of 3-(3,4-dihydroxyphenyl)-2-propenoic acid triacontyl ester per 100 mg of extract.

In some embodiments, the rice bran extract comprises about 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500 μg of bombiprenone, per 100 mg of extract.

In some embodiments, the rice bran extract comprises about 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, or 2000 μg of glycerol 1,2-di-(9Z,12Z-octadecadienoate), per 100 mg of extract.

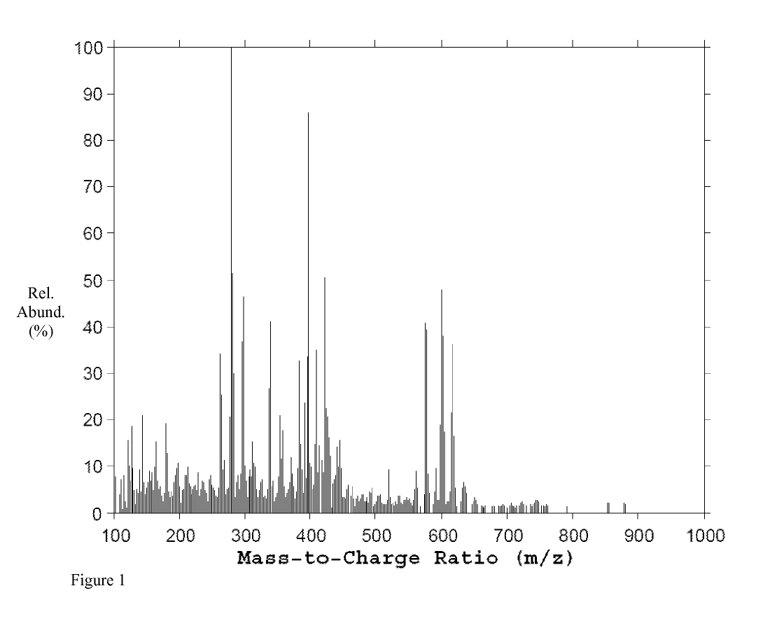

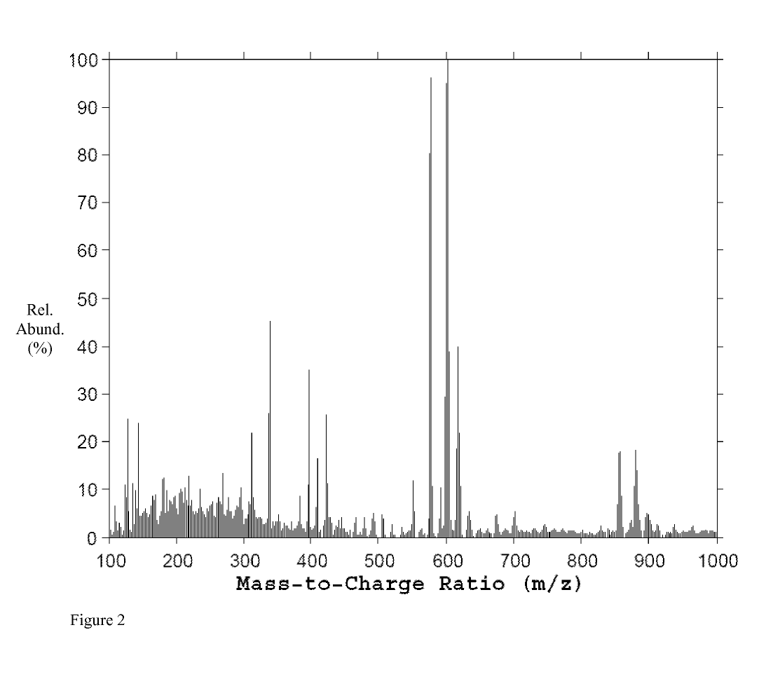

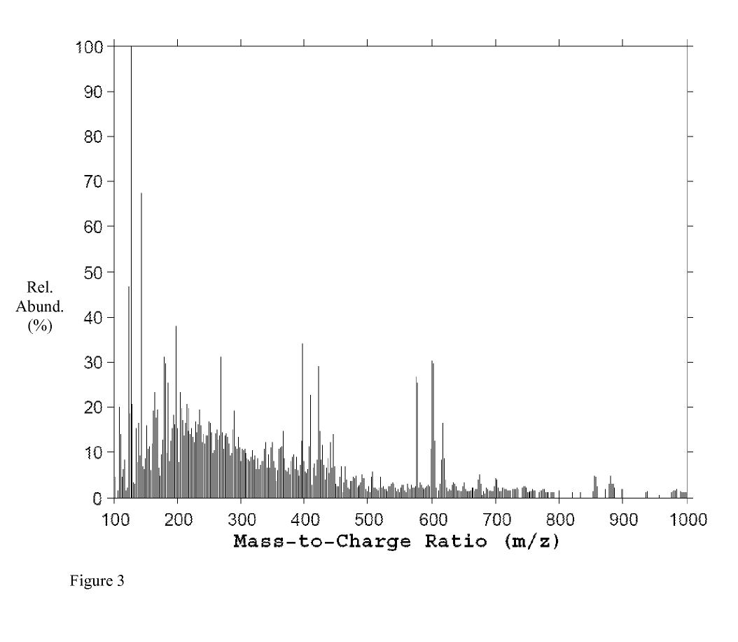

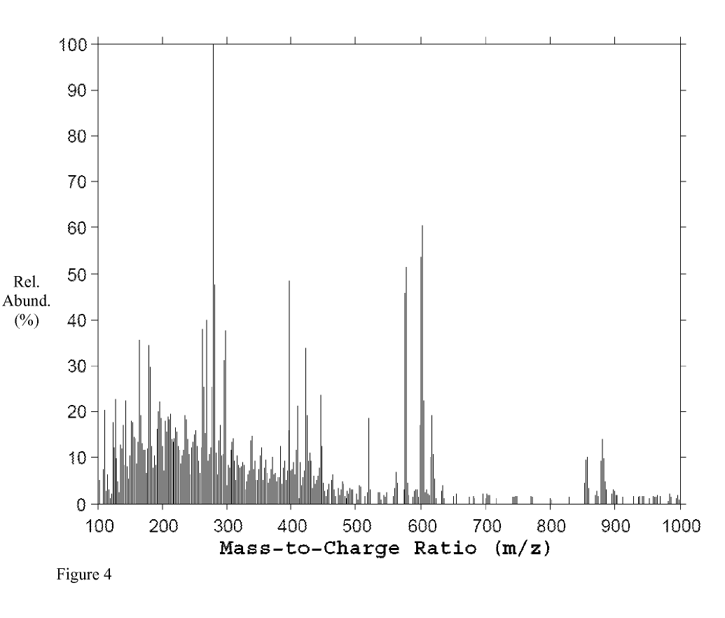

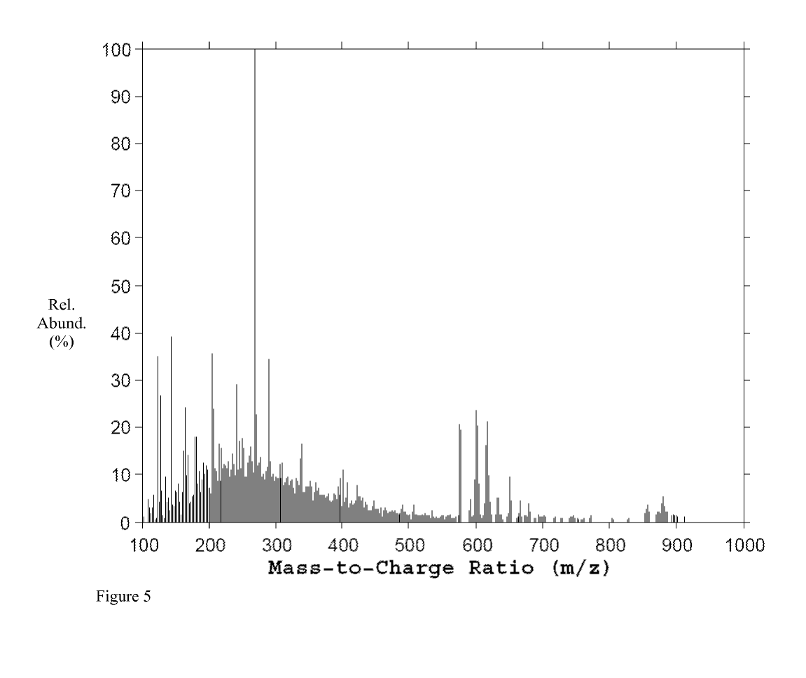

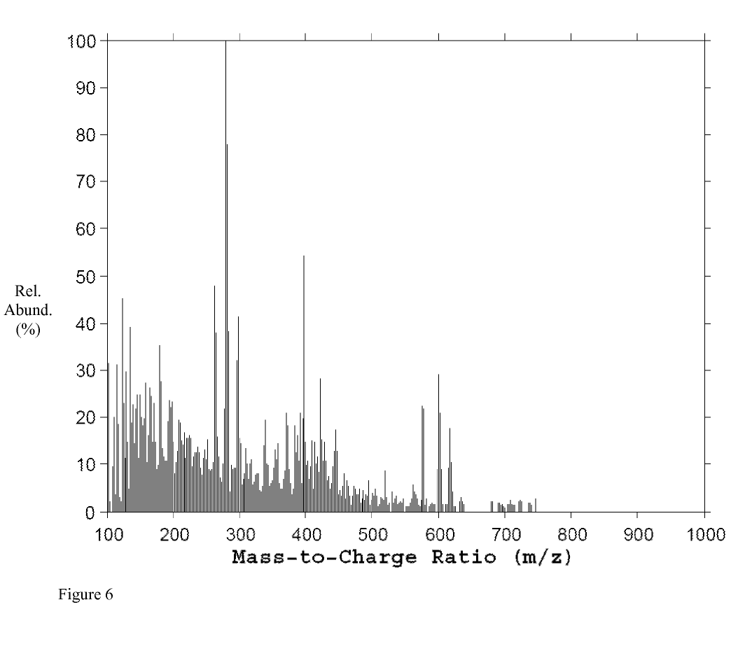

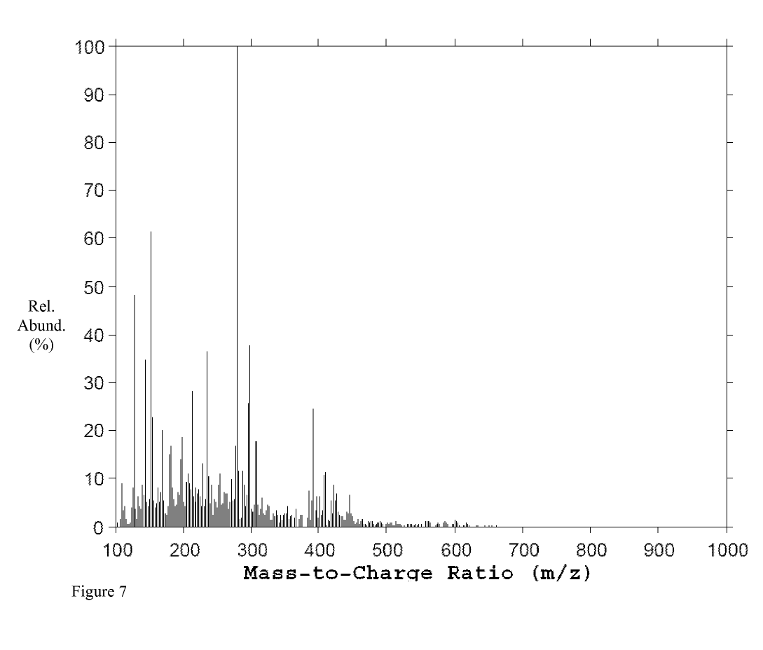

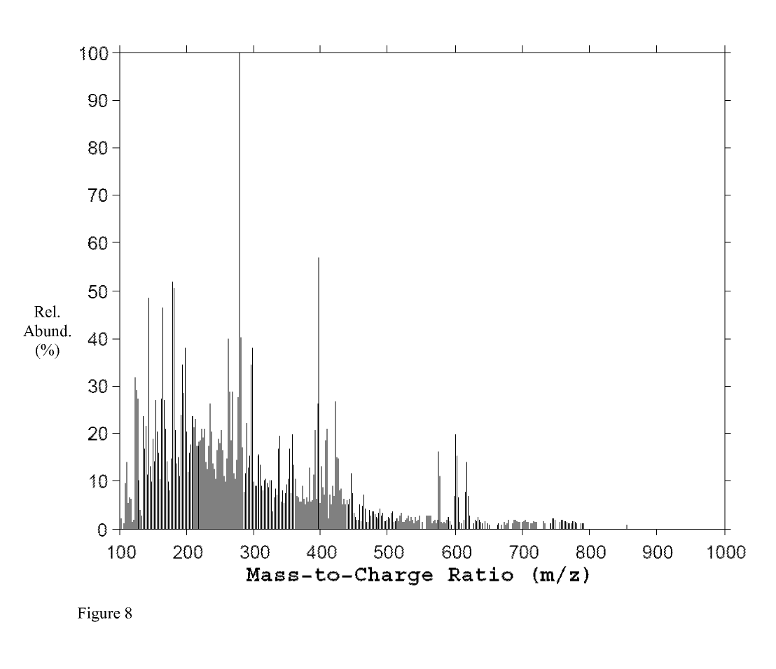

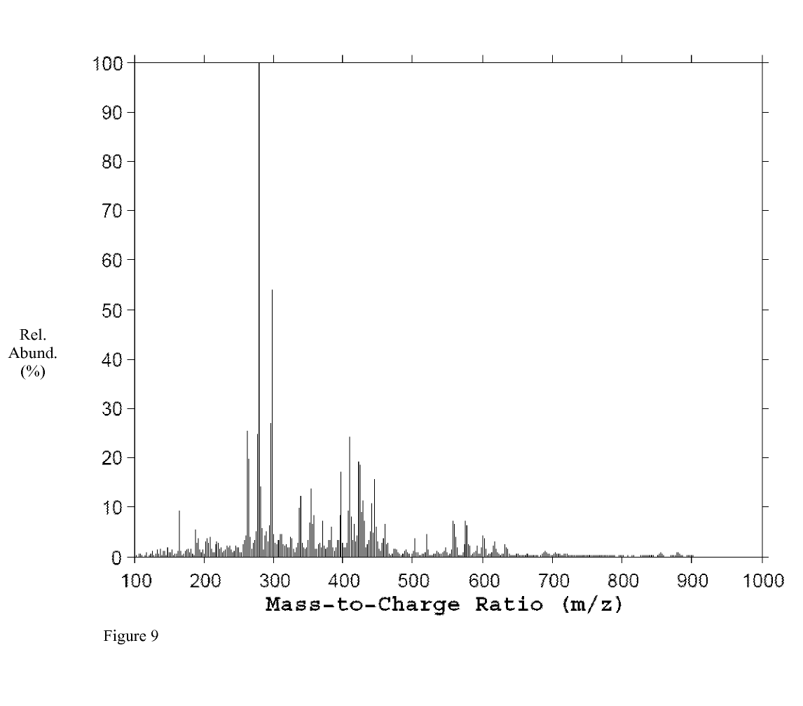

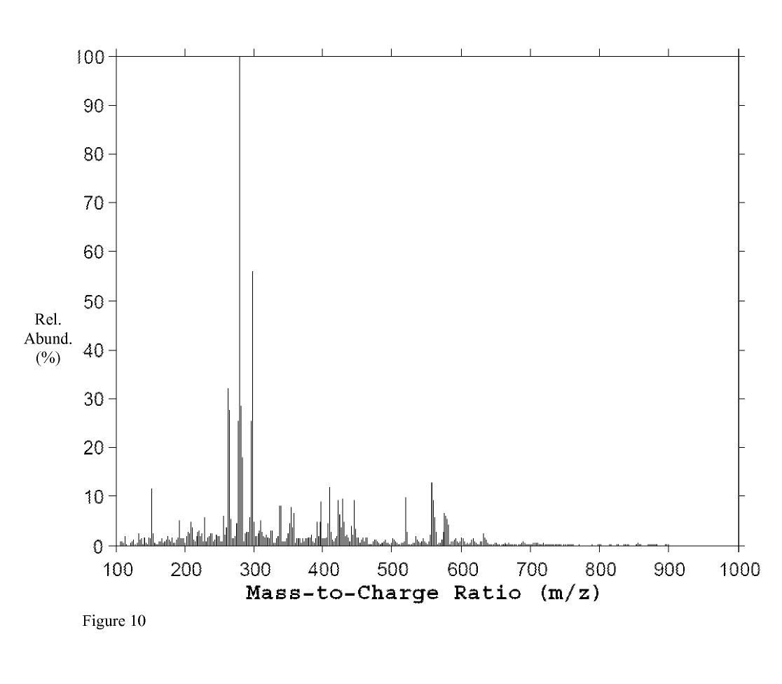

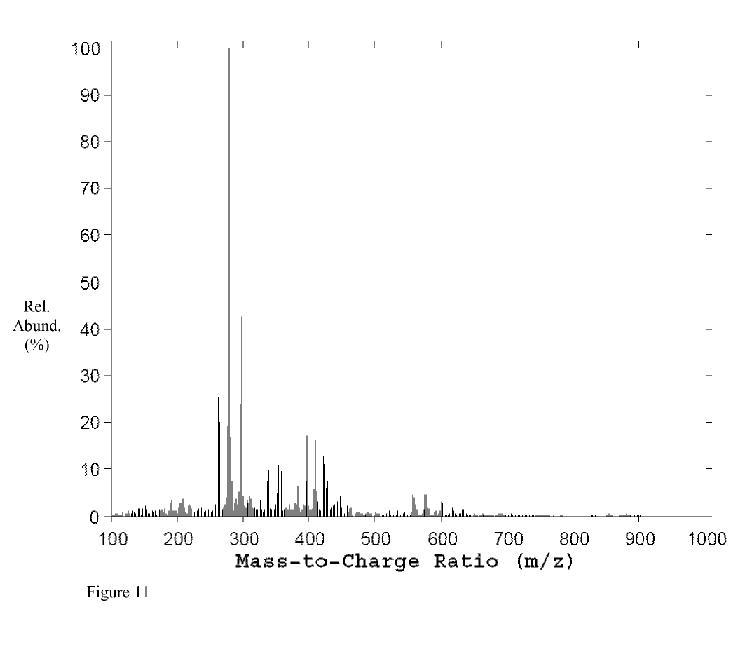

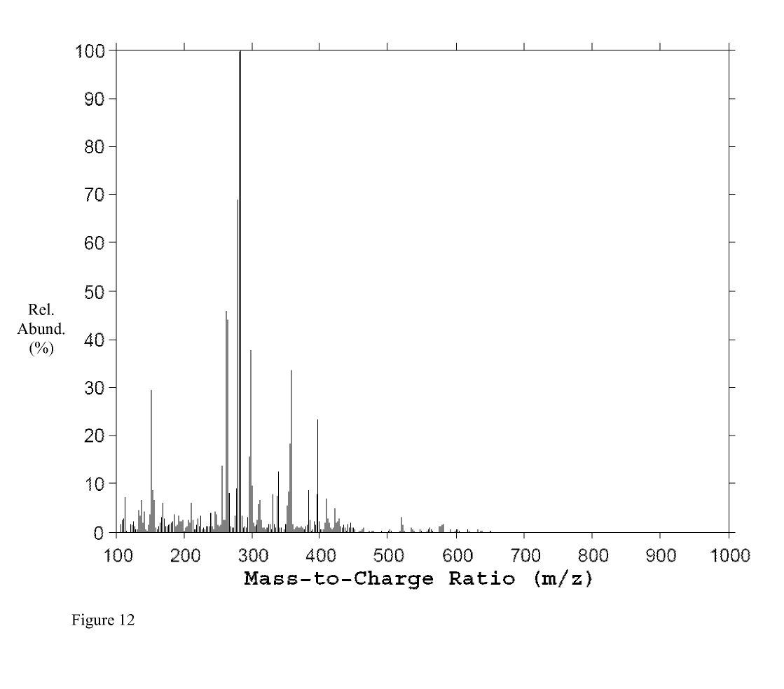

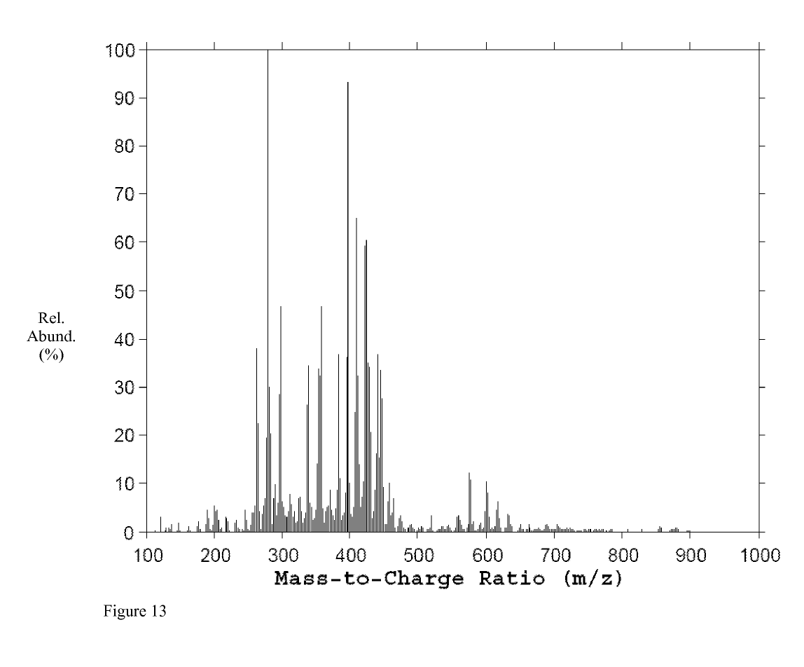

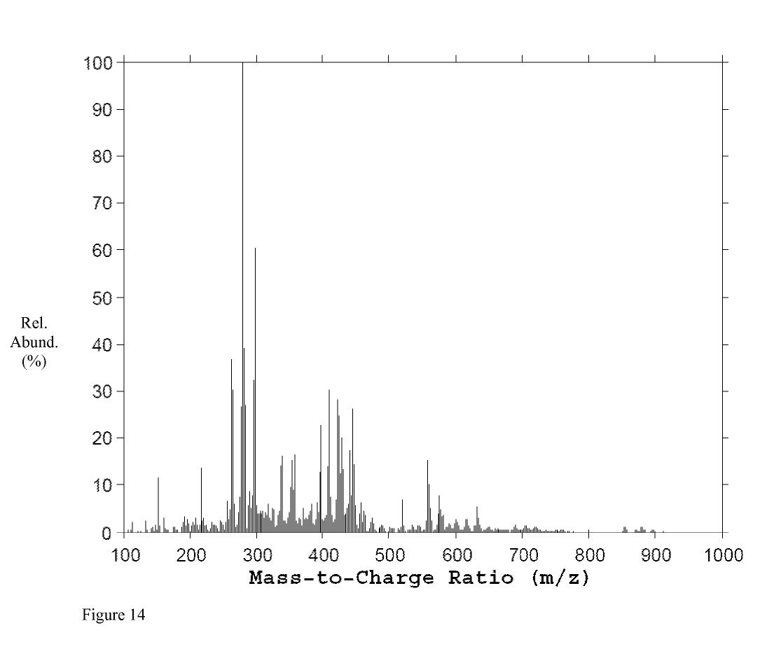

In some embodiments, the present invention relates to a rice bran extract, such as any of the aforementioned extracts, having a fraction comprising a Direct Analysis in Real Time (DART) mass spectrometry chromatogram of any of FIGS. 1 to 14.

In some embodiments, the rice bran extract has a glucose uptake stimulation greater than a glucose uptake stimulation of 200 nM insulin. In some embodiments, the glucose uptake stimulation of the extract is 0.5 to 5 times greater than the glucose uptake stimulation of 200 nM insulin. In some embodiments, the glucose uptake stimulation of the extract is 0.5 to 3.5 times greater than the glucose uptake stimulation of 200 nM insulin. In some embodiments, the glucose uptake stimulation of the extract is 0.7 to 3.1 times greater than the glucose uptake stimulation of 200 nM insulin. In other embodiments, the glucose uptake stimulation of the extract is more than 3 times greater than the glucose uptake stimulation of 200 nM insulin. In other embodiments, the glucose uptake stimulation of the extract is about 3 times greater than the glucose uptake stimulation of 200 nM insulin.

In another embodiment, the extract has a glucose uptake stimulation greater than a glucose uptake stimulation of control. In some embodiments, the extract glucose uptake stimulation is more than 1 times greater than the glucose uptake stimulation of control. In other embodiments, the extract glucose uptake stimulation is 1 to 10 times greater than the glucose uptake stimulation of control. In other embodiments, the extract glucose uptake stimulation is 2 to 7 times greater than the glucose uptake stimulation of control. In other embodiments, the extract glucose uptake stimulation is about 6 times greater than the glucose uptake stimulation of control.

In some embodiments, the extract has a glucose uptake stimulation of 100 to 3000 counts per minute (cpm). In other embodiments, the extract has a glucose uptake stimulation of 100 to 1000 cpm. In some embodiments, the concentration of the extract is 5 to 2000 μg/mL and the glucose uptake stimulation of 100 to 3000 cpm or 100 to 1000 cpm. In other embodiments, the concentration of extract is 10 to 1000 μg/mL. In other embodiments, the concentration of extract is 10, 50, 250 or 1000 μg/mL.

In some embodiments, the rice bran extract has an IC₅₀ value for FABP4 inhibition of less than 2000 μg/mL. In other embodiments, the IC₅₀ value for FABP4 inhibition is from 25 to 2000 μg/mL, from 25 to 1000 μg/mL, or from 25 to 500 μg/mL. In some embodiments, the IC₅₀ value for FABP4 inhibition is from 100 to 1000 μg/mL. In other embodiments, the IC₅₀ value for FABP4 inhibition is about 100, 200, 300, 400, 500, 600, 700, 800, 900 or 1000 μg/mL.

Another aspect of the invention relates to a rice bran extract prepared by a process comprising the following steps:
a) providing a stabilized rice bran feedstock, and
b) extracting the feedstock.
   In some embodiments, the extracting step is an aqueous ethanol extraction, while in other embodiments, the extracting step is supercritical carbon dioxide extraction. In some embodiments, the aqueous ethanol is about 10 to 99% ethanol. In other embodiments, the aqueous ethanol is about 20 to 90% ethanol. In other embodiments, the aqueous ethanol is about 20, 30, 40, 50, 60, 70, 80 or 90% ethanol. In other embodiments, the aqueous ethanol is about 40 to 80% ethanol. In some embodiments, the aqueous ethanol extraction is performed at a temperature of about 20 to 80° C. In other embodiments, the extraction is performed at a temperature of about 30 to 70° C. In other embodiments, the temperature is about 40 to 60° C. In other embodiments, the temperature is about 30, 40, 50, 60, or 70° C.

In some embodiments, the supercritical carbon dioxide extraction is performed at a temperature of about 20 to 100° C. In other embodiments, the temperature is about 30 to 90° C., or 40 to 80° C. In other embodiments, the temperature is about 40, 50, 60, 70 or 80° C. In some embodiments, the pressure of the super critical carbon dioxide extraction is about 200 to 800 bar. In other embodiments, the pressure is about 200 to 600 bar. In other embodiments, the pressure is about 300 to 500 bar. In some embodiments, the pressure is about 300 bar, 400 bar, or 500 bar.

Pharmaceutical Compositions

In some aspects of the invention, pharmaceutical formulations comprising any of the aforementioned and at least one pharmaceutically acceptable carrier are provided.

Compositions of the disclosure comprise extracts of stabilized rice bran in forms such as a paste, powder, oils, liquids, suspensions, solutions, ointments, or other forms, comprising, one or more fractions or sub-fractions to be used as dietary supplements, nutraceuticals, or such other preparations that may be used to prevent or treat various human ailments. The extracts can be processed to produce such consumable items, for example, by mixing them into a food product, in a capsule or tablet, or providing the paste itself for use as a dietary supplement, with sweeteners or flavors added as appropriate. Accordingly, such preparations may include, but are not limited to, rice bran extract preparations for oral delivery in the form of tablets, capsules, lozenges, liquids, emulsions, dry flowable powders and rapid dissolve tablet. Based on the anti-allergic activities described herein, patients would be expected to benefit from daily dosages in the range of from about 50 mgs to about 1000 mg. For example, a lozenge comprising about 50, 55, 60, 65, 70, 75, 80, 85, 90, 95, 100, 105, 110, 115, 120, 125, 130, 135, 140, 145, 150, 160, 170, 180, 190, 200, 210, 220, 230, 240, or 250 mg of the extract can be administered once or twice a day to a subject as a prophylactic. Alternatively, in response to a severe allergic reaction, two lozenges may be needed every 4 to 6 hours.

In one embodiment, a dry extracted rice bran composition is mixed with a suitable solvent, such as but not limited to water or ethyl alcohol, along with a suitable food-grade material using a high shear mixer and then spray air-dried using conventional techniques to produce a powder having grains of very small rice bran extract particles combined with a food-grade carrier.

In a particular example, rice bran extract composition is mixed with about twice its weight of a food-grade carrier such as maltodextrin having a particle size of between 100 to about 150 micrometers and an ethyl alcohol solvent using a high shear mixer. Inert carriers, such as silica, preferably having an average particle size on the order of about 1 to about 50 micrometers, can be added to improve the flow of the final powder that is formed. Preferably, such additions are up to 2% by weight of the mixture. The amount of ethyl alcohol used is preferably the minimum needed to form a solution with a viscosity appropriate for spray air-drying. Typical amounts are in the range of between about 5 to about 10 liters per kilogram of extracted material. The solution of extract, maltodextrin and ethyl alcohol is spray air-dried to generate a powder with an average particle size comparable to that of the starting carrier material.

In another embodiment, an extract and food-grade carrier, such as magnesium carbonate, a whey protein, or maltodextrin are dry mixed, followed by mixing in a high shear mixer containing a suitable solvent, such as water or ethyl alcohol. The mixture is then dried via freeze drying or refractive window drying. In a particular example, extract material is combined with food grade material about one and one-half times by weight of the extract, such as magnesium carbonate having an average particle size of about 20 to 200 micrometers. Inert carriers such as silica having a particle size of about 1 to about 50 micrometers can be added, preferably in an amount up to 2% by weight of the mixture, to improve the flow of the mixture. The magnesium carbonate and silica are then dry mixed in a high speed mixer, similar to a food processor-type of mixer, operating at 100's of rpm. The extract is then heated until it flows like a heavy oil. Preferably, it is heated to about 50° C. The heated extract is then added to the magnesium carbonate and silica powder mixture that is being mixed in the high shear mixer. The mixing is continued preferably until the particle sizes are in the range of between about 250 micrometers to about 1 millimeter. Between about 2 to about 10 liters of cold water (preferably at about 4° C.) per kilogram of extract is introduced into a high shear mixer. The mixture of extract, magnesium carbonate, and silica is introduced slowly or incrementally into the high shear mixer while mixing. An emulsifying agent such as carboxymethylcellulose or lecithin can also be added to the mixture if needed. Sweetening agents such as Sucralose or Acesulfame K up to about 5% by weight can also be added at this stage if desired. Alternatively, extract of Stevia rebaudiana, a very sweet-tasting dietary supplement, can be added instead of or in conjunction with a specific sweetening agent (for simplicity, Stevia will be referred to herein as a sweetening agent). After mixing is completed, the mixture is dried using freeze-drying or refractive window drying. The resulting dry flowable powder of extract, magnesium carbonate, silica and optional emulsifying agent and optional sweetener has an average particle size comparable to that of the starting carrier and a predetermined extract.

According to another embodiment, an extract is combined with approximately an equal weight of food-grade carrier such as whey protein, preferably having a particle size of between about 200 to about 1000 micrometers. Inert carriers such as silica having a particle size of between about 1 to about 50 micrometers, or carboxymethylcellulose having a particle size of between about 10 to about 100 micrometers can be added to improve the flow of the mixture. Preferably, an inert carrier addition is no more than about 2% by weight of the mixture. The whey protein and inert ingredient are then dry mixed in a food processor-type of mixer that operates over 100 rpm. The extract can be heated until it flows like a heavy oil (preferably heated to about 50° C.). The heated extract is then added incrementally to the whey protein and inert carrier that is being mixed in the food processor-type mixer. The mixing of the extract and the whey protein and inert carrier is continued until the particle sizes are in the range of about 250 micrometers to about 1 millimeter. Next, 2 to 10 liters of cold water (preferably at about 4° C.) per kilogram of the paste mixture is introduced in a high shear mixer. The mixture of extract, whey protein, and inert carrier is introduced incrementally into the cold water containing high shear mixer while mixing. Sweetening agents or other taste additives of up to about 5% by weight can be added at this stage if desired. After mixing is completed, the mixture is dried using freeze drying or refractive window drying. The resulting dry flowable powder of extract, whey protein, inert carrier and optional sweetener has a particle size of about 150 to about 700 micrometers and an unique predetermined extract.

In the embodiments where the extract is to be included into an oral fast dissolve tablet as described in U.S. Pat. No. 5,298,261, the unique extract can be used “neat,” that is, without any additional components which are added later in the tablet forming process as described in the patent cited. This method obviates the necessity to take the extract to a dry flowable powder that is then used to make the tablet.

Once a dry extract powder is obtained, such as by the methods discussed herein, it can be distributed for use, e.g., as a dietary supplement or for other uses. In a particular embodiment, the novel extract powder is mixed with other ingredients to form a tableting composition of powder that can be formed into tablets. The tableting powder is first wet with a solvent comprising alcohol, alcohol and water, or other suitable solvents in an amount sufficient to form a thick doughy consistency. Suitable alcohols include, but not limited to, ethyl alcohol, isopropyl alcohol, denatured ethyl alcohol containing isopropyl alcohol, acetone, and denatured ethyl alcohol containing acetone. The resulting paste is then pressed into a tablet mold. An automated tablet molding system, such as described in U.S. Pat. No. 5,407,339, can be used. The tablets can then be removed from the mold and dried, preferably by air-drying for at least several hours at a temperature high enough to drive off the solvent used to wet the tableting powder mixture, typically between about 70° to about 85° C. The dried tablet can then be packaged for distribution

Compositions can be in the form of a paste, resin, oil, powder or liquid. Liquid preparations for oral administration may take the form of, for example, solutions, syrups or suspensions, or they may be presented as a dry product for reconstitution with water or other suitable vehicle prior to administration. Such liquid preparations may be prepared by conventional means with pharmaceutically acceptable additives such as suspending agents (e.g., sorbitol syrup, methyl cellulose, or hydrogenated edible fats); emulsifying agents (e.g., lecithin or acacia); non-aqueous vehicles (e.g., almond oil, oily esters or ethyl alcohol); preservatives (e.g., methyl or propyl p-hyroxybenzoates or sorbic acid); and artificial or natural colors and/or sweeteners. Compositions of the liquid preparations can be administered to humans or animals in pharmaceutical carriers known to those skilled in the art. Such pharmaceutical carriers include, but are not limited to, capsules, lozenges, syrups, sprays, rinses, and mouthwash.

Dry powder compositions may be prepared according to methods disclosed herein and by other methods known to those skilled in the art such as, but not limited to, spray air drying, freeze drying, vacuum drying, and refractive window drying. The combined dry powder compositions can be incorporated into a pharmaceutical carrier such, but not limited to, tablets or capsules, or reconstituted in a beverage such as a tea.

The described extracts may be combined with extracts from other plants such as, but not limited to, varieties of Gymnemia, turmeric, Boswellia, Guarana, cherry, lettuce, Echinacea, piper betel leaf, Areca catechu, Muira puama, ginger, willow, suma, kava, horny goat weed, Ginkgo biloba, mate, garlic, puncture vine, arctic root Astragalus, eucommia, gastropodia, and uncaria, or pharmaceutical or nutraceutical agents.

A tableting powder can be formed by adding about 1 to 40% by weight of the powdered extract, with between 30 to about 80% by weight of a dry water-dispersible absorbent such as, but not limited to, lactose. Other dry additives such as, but not limited to, one or more sweetener, flavoring and/or coloring agents, a binder such as acacia or gum arabic, a lubricant, a disintegrant, and a buffer can also be added to the tableting powder. The dry ingredients are screened to a particle size of between about 50 to about 150 mesh. Preferably, the dry ingredients are screened to a particle size of between about 80 to about 100 mesh.

Preferably, the tablet exhibits rapid dissolution or disintegration in the oral cavity. The tablet is preferably a homogeneous composition that dissolves or disintegrates rapidly in the oral cavity to release the extract content over a period of about 2 seconds or less than 60 seconds or more, preferably about 3 to about 45 seconds, and most preferably between about 5 to about 15 seconds.

Various rapid-dissolve tablet formulations known in the art can be used. Representative formulations are disclosed, for example, in U.S. Pat. Nos. 5,464,632; 6,106,861; 6,221,392; 5,298,261; and 6,200,604; the entire contents of each are expressly incorporated by reference herein. For example, U.S. Pat. No. 5,298,261 teaches a freeze-drying process. This process involves the use of freezing and then drying under a vacuum to remove water by sublimation. Preferred ingredients include hydroxyethylcellulose, such as Natrosol from Hercules Chemical Company, added to between 0.1 and 1.5%. Additional components include maltodextrin (Maltrin, M-500) at between 1 and 5%. These amounts are solubilized in water and used as a starting mixture to which is added the rice bran extraction composition, along with flavors, sweeteners such as Sucralose or Acesulfame K, and emulsifiers such as BeFlora and BeFloraPlus which are extracts of mung bean. A particularly preferred tableting composition or powder contains about 10 to 60% by of the extract powder and about 30% to about 60% of a water-soluble diluent.

In a preferred implementation, the tableting powder is made by mixing in a dry powdered form the various components as described above, e.g., active ingredient (extract), diluent, sweetening additive, and flavoring, etc. An overage in the range of about 10% to about 15% of the active extract can be added to compensate for losses during subsequent tablet processing. The mixture is then sifted through a sieve with a mesh size preferably in the range of about 80 mesh to about 100 mesh to ensure a generally uniform composition of particles.

The tablet can be of any desired size, shape, weight, or consistency. The total weight of the extract in the form of a dry flowable powder in a single oral dosage is typically in the range of about 40 mg to about 1000 mg. The tablet is intended to dissolve in the mouth and should therefore not be of a shape that encourages the tablet to be swallowed. The larger the tablet, the less it is likely to be accidentally swallowed, but the longer it will take to dissolve or disintegrate. In a preferred form, the tablet is a disk or wafer of about 0.15 inch to about 0.5 inch in diameter and about 0.08 inch to about 0.2 inch in thickness, and has a weight of between about 160 mg to about 1,500 mg. In addition to disk, wafer or coin shapes, the tablet can be in the form of a cylinder, sphere, cube, or other shapes.

Compositions of unique extract compositions may also comprise extract compositions in an amount between about 10 mg and about 2000 mg per dose.

### Methods of Treatment

Another aspect of the invention relates to a method of stimulating glucose uptake comprising administering to a subject in need thereof an effective amount of any of the aforementioned rice bran extracts or pharmaceutical compositions.

Another aspect of the invention relates to a method if inhibiting FABP4 binding comprising administering to a subject in need thereof an effective amount of any of the aforementioned rice bran extracts or pharmaceutical compositions. In some embodiments, the subject has hyperglycemia. In other embodiments, the subject has diabetes. In other embodiments, the subject has type 1 diabetes, while in other embodiments, the subject has type 2 diabetes. In other embodiments, the subject has obesity and related metabolic disorders.

In some embodiments, the subject is a mammal, such as a primate, for example a human.

### Exemplification

### Methods

### A. Stabilized Rice Bran Feedstocks

Stabilized Rice Bran (SRB) was supplied by Nutracea Inc., USA and stored at room temperature. The SRB was sieved through a 140 mesh screen (100 μm) prior to use.

### B. Stabilized Rice Bran Extract Preparation

### 1. Solvent Extraction

A 10 g of SRB was extracted in a flask with 150 mL of organic solvents used for plant materials. Solvents of different concentration of ethanol in water like water, 20% (v/v) ethanol, 40% ethanol, 60% ethanol, and 80% ethanol and 100% ethanol were used. The extraction was performed in two, 2-hr stages at temperatures of 20 to 60° C. The combined extracts were filtered through Fisher P4 filter paper with a pore size of 4-8 μm, and centrifuge at 2000 rpm for 20 minutes. The supernatants were collected and evaporated to dryness at 50° C. in a vacuum oven for overnight.

### 2. Supercritical Carbon Dioxide Extraction

Supercritical Carbon Dioxide (SCCO) extraction experiments were performed using a SFT 250 (Supercritical Fluid Technologies, Inc., Newark, Del.) which is designed for pressures and temperatures up to 690 bar and 200° C., respectively. The apparatus consisted of three modules; an oven, a pump and control, and collection module. The pump module was equipped with a compressed air-driven pump with constant flow capacity of 300 mL min⁻¹, while the collection module was a 40 mL glass vial sealed with caps and septa for the recovery of extracted products. The extraction vessel pressure and temperature are monitored and controlled within ±3 bar and ±1° C.

A sample, 30 g, of SRB powder with mesh sizes above 105 μm (measured using a 140 mesh screen) was loaded into a 100 mL extraction vessel for each experiment. Glass wool was placed at the two ends of the column to avoid any possible carryover of solid material. The oven was preheated to the desired temperature before the packed vessel was loaded. The system was closed and pressurized to the desired extraction pressure using the air-driven liquid pump and equilibrated for ˜3 min. A sampling vial (40 mL) was weighed and connected to the sampling port. The extraction was started by flowing CO₂ at a rate of ˜10 SLPM (19 g/min). The yield was defined to be the weight ratio of total exacts to the feed of raw material. The yield was defined as the weight percentage of the oil extracted with respect to the initial charge of the raw material in the extractor. A full factorial extraction design was adopted varying the temperature from 40-80° C. and from 80-500 bar.

### C. DART TOF-MS Characterization of Extracts

A Jeol DART AccuTOF-MS (Model JMS-T100LC; Jeol USA, Peabody, Mass.) was used for chemical characterization of compounds in SRB extracts. The DART settings were loaded as follows: DART needle voltage=3000V; Electrode 1 voltage=150V; Electrode 2 voltage=250 V; Temperature=250° C.; He Flow Rate=2.52 LPM. The following AccuTOF mass spectrometer settings were loaded: Ring Lens voltage=5 V; Orifice 1 voltage=10 V; Orifice 2 voltage=5 V; Peaks voltage=1000 V (for resolution between 100-1000 amu); Orifice 1 temperature was turned off. The samples were introduced by placing the closed end of a borosilicate glass capillary tube into the SRB extracts, and the coated capillary tube was placed into the DipIT™ sample holder providing a uniform and constant surface exposure for ionization in the He plasma. The SRB extract was allowed to remain in the He plasma stream until signal was observed in the total-ion-chromatogram (TIC). The sample was removed and the TIC was brought down to baseline levels before the next sample was introduced. A polyethylene glycol 600 (Ultra Chemicals, Kingston, R.I.) was used as an internal calibration standard giving mass peaks throughout the desired range of 100-1000 amu. The DART mass spectra of each SRB extract was searched against a proprietary chemical database and used to identify many of the compounds present in the extracts. Search criteria were held to the [M+H]⁺ ions to within 10 mmu of the calculated masses. The identified compounds are reported with greater than 90% confidence. DART mass spectra of extracts 1 to 14 are shown in FIGS. 1 to 14, respectively, with the X-axis showing the mass distribution (100-1000 m/z [M+H+]) and the y-axis showing the relative abundances of each chemical species detected.
D. Glucose Uptake

1. [1,2-³H]2-Deoxy-D-glucose (2-deoxyglucose) Uptake: Cells, 3T3-L adipocytes, were grown and differentiated as described below. Prior to [³H]2-deoxyglucose uptake, cells were switched to DMEM with 0.1% bovine serum albumin for 6 h. The [³H]2-deoxyglucose uptake was assayed as described (D. R. Cooper, J. E. Watson, N. Patel, P. Illingworth, M. Cevedo-Duncan, J. Goodnight, C. E. Chalfant, and H. Mischak, 1999. Ectopic expression of protein kinase CbetaII, -delta, and -epsilon, but not-betaI or -zeta, provide for insulin stimulation of glucose uptake in NIH-3T3 cells. Arch. Biochem. Biophys., 372:69-79; T. P Ciraldi, O. G. Kolterman, and J. M. Olesky, 1981. Mechanism of the postreceptor defect in insulin action in human obesity: decrease in glucose transport system activity. J. Clin Invest., 68:875-880.). Cells were preincubated 10 min with Dulbecco's phosphate buffered saline (DPBS) with 1% bovine serum albumin (BSA), insulin (1-100 nM) or the vehicle, DPBS+BSA, was added and cells were incubated an additional 20 min at 37° C. Uptake was measured by the addition of 10 nmol of [³H] 2-deoxyglucose (50-150 μCi/μmol) and followed by incubation for 6 min at 37° C. The uptake was terminated by aspiration of media and cell monolayers were washed three times with cold DPBS. Cells were lysed with 1 ml of 1% (w/v) SDS, and radioactivity determined by liquid scintillation counting. The 2-Deoxyglucose uptake refers to transport of the analogue across the plasma membrane operating in tandem with its phosphorylation by hexokinase.

2. 3-0-Fmethyl-¹⁴C] glucose Uptake: For 3-0-methylglucose uptake, cells are pre-incubated in the transport buffer with insulin (10 nM) added for 30 min prior to addition of 32 μM 3-0-[methyl-4C] glucose (50 mCi/mmol) for 0.5 or 1 min, and stopped as described above (R. R. Whitesell and J. Gliemann, 1979. Kinetic parameters of transport of 3-O-methylglucose and glucose in adipocytes. J. Biol. Chem., 254:5276-5283). Control studies indicate that under these conditions, 3-0-methylglucose uptake is linear during the first minute of uptake.

3. Cytochalasin B Inhibition Assays: Possible impacts on cytoskeletal activity by the SRB extracts that could affect glucose uptake were evaluated using methods of Estensen and Plagemann (R. D. Estensen and P. G. W. Plagemann, 1972. Cytochalasin B: Inhibition of glucose and glucosamine transport. Proc. Natl. Acad. Sci. USA 69: 1430-1434).

### E. Receptor and Transporter Expression Studies

1. Insulin Receptor Expression: Extracts were examined for expression of insulin receptors, GLUT4 translocator (D. R. Cooper, J. E. Watson, N. Patel, P. Illingworth, M. Cevedo-Duncan, J. Goodnight, C. E. Chalfant, and H. Mischak, 2001. Ectopic expression of protein kinase CbetaII, -delta, and -epsilon, but not -betaI or -zeta, provide for insulin stimulation of glucose uptake in NIH-3T3 cells. Arch. Biochem. Biophys., 372:69-79; C. E. Chalfant, S. Ohno, Y. Konno, A. A. Fisher, L. D. Bisnauth, J. E. Watson, and D. R. Cooper, 1996. A carboxy-terminal deletion mutant of protein kinase C beta II inhibits insulin-stimulated 2-deoxyglucose uptake in L6 rat skeletal muscle cells. Mol. Endocrinol., 10:1273-1281; N. A. Patel, C. E. Chalfant, J. E. Watson, J. R. Wyatt, N. M. Dean, D. C. Eichler, and D. R. Cooper, 2001. Insulin regulates alternative splicing of protein kinase C beta II through a phosphatidylinositol 3-kinase-dependent pathway involving the nuclear serine/arginine-rich splicing factor, SRp4O, in skeletal muscle cells. J. Biol. Chem., 276:22648-22654), IRS-1 activity and PI-3 Kinase/AKT activity using Western blot analysis.

2. Phosphorvlation State of IRS-1 and AKT: The phosphorylation state of IRS-1 and AKT were determined as described by Patel et al. (N. A. Patel, C. E. Chalfant, J. E. Watson, J. R. Wyatt, N. M. Dean, D. C. Eichler, and D. R. Cooper, 2001. Insulin regulates alternative splicing of protein kinase C beta II through a phosphatidylinositol 3-kinase-dependent pathway involving the nuclear serine/arginine-rich splicing factor, SRp40, in skeletal muscle cells. J. Biol. Chem., 276:22648-22654).

3. Translocation of GLUT4 from the ER to the cell surface: Translocation of GLUT4 from the ER to the plasma membrane was assessed by fluorescence microscopy using antibodies to GLUT4 with a fluorescent tag.

F. Zucker Rat Obese Model

Studies were designed to examine if SRB extracts CR reduce hyperglycemia and other aspects of type 2 diabetes in the Zucker obese rat model with the Zucker lean rat serving as a control. The Zucker-obese rat is hyperglycemic and considered a good rodent model of type 2 non-insulin-dependent diabetes mellitus (NIDDM). Both Zucker-obese and Zucker-lean rats are glucose intolerant at 8 weeks of age. The Zucker-lean rat does not become hyperglycemic but is hyperinsulinemic through 32 wk of age. All Zucker-obese rats become hyperglycemic by 8 weeks of age.

Zucker-obese, Zucker-lean, and F344 rats were used. Groups of 10 Zucker obese, Zucker lean or F344 rats were started on either control or CR diet and followed for 2 or 4 months. The animals were housed and maintained at the fully accredited AAALAC animal facilities at USFCOM in Tampa, Fla. in accordance with Institutional Guidelines. Animal handling was approved by the Laboratory Animal Medical Ethics Committee, USFCOM. Euthanasia was performed with sodium pentobarbital as approved by the LAMEC and defined in the approved IACUC.

Animals entered the study at 10 weeks of age and fed normal rodent chow and given tap water ad libitum. Glucose and insulin level were monitored in the rats and after 4 weeks of extract administration and rats were given glucose and an insulin challenges to examine for changes in glucose tolerance and insulin tolerance. Furthermore cell signaling mechanisms in adipocytes were assessed in isolated tissues from the rats at the end of the experiment. Pancreata was collected from each euthanized rat and processed for light (LM) and electron microscopic (EM) analysis. Tissues for LM were fixed with 4% paraformaldehyde/PBS, processed into paraffin and stained with H&E for routine histology/pathology. Some paraffin slides were stained with DTZ to identify β-cells and some with ApoTag to determine apoptosis of islet cells. Double-labeled immunostaining for β-cells and apoptosis were performed to detect β-cell destruction. Tissues for EM were fixed with 5% (v/v) gluteraldehyde and routinely processed into plastic resin. Thick sections were stained with Toluidine Blue (light microscopy) and thin section with UA/LC (electron microscopy).

Animal Monitoring: At the beginning of the study, all rats were weighed and non-fasting blood glucose recorded from tail vein blood determined by FreeStyle™ glucometer and test strips. Daily, all rats were observed for any visible changes in their general condition and non-fasting blood glucose concentrations were determined with the FreeStyle™ system. Weekly, all rats were weighed and food consumption monitored. Urine glucose and insulin levels were determined following 24 h in metabolic cages every 2 weeks after the initiation of CR treatment. General condition, body weights, blood and urine glucose concentrations and monthly urine insulin concentrations were recorded. Glucose tolerance tests and insulin tolerance tests were conducted at bi-weekly intervals.

### G. FABP4 Inhibition Studies

Fatty Acid Binding Protein 4 (FABP4) inhibition was determined using the Fatty Acid Binding Protein 4 (FABP4) Inhibitor/Ligand Screening Kit (Cayman, Ann Arbor, Mich.). The assay uses a 96-well plate format that includes positive and negative controls, serial dilutions of a standard (arachidonic acid), and extracts that either receive detection reagent (detection wells) or do not receive detection reagent (undetected wells). Potential inhibitors/ligands of the FABP4 protein were incubated to FABP4 in assay buffer for 15 minutes at room temperature. Arachidonic acid was used as a known inhibitor standard for comparison. The positive control wells received no inhibitor/ligand (i.e., no arachidonic acid or extract) and the negative control wells received no FABP4. The extracts, in solution, were then exposed to a developer that will fluoresce when bound to FABP4. If FABP4 is inhibited, reduction in fluorescence yield is observed. Fluorescence was quantified using a Synergy 4 plate reader that is tuned to excitation/emission wavelengths of 370 nm and 475 nm, respectively. The fluorescence of the negative controls was subtracted from the positive control wells, and the fluorescence from the “undetected” wells was subtracted from the corresponding “detected” wells. An IC₅₀ value was determined based on the percent fluorescence of the corrected extract wells relative to the corrected positive controls.

### Results

### A. Glucose Uptake

Table 1 summarizes the dose-dependent uptake of [1,2-³H]2-Deoxy-D-glucose (2-deoxyglucose) uptake in 3T3-L1 cells in the presence of varying concentrations of SRB Extracts 1-10, and the dose-dependent uptake of 3-O-methylglucose in 3T3-L1 cells in the presence of varying concentrations of Extracts 11-15.

TABLE 1
Dose-dependent uptake of [1,2-³H]2-Deoxy-D-glucose (2-deoxyglucose)
uptake in 3T3-L1 cells in the presence of varying levels of SRB Extracts
1-10, and the dose-dependent uptake of 3-O-methylglucose in 3T3-L1
cells in the presence of varying levels of Extracts 11-15 presented as
maximum cpms.

| | Extract | |
|---|---|---|
| Extract | (μg mL⁻¹) | CPM |
| Control | | 131 |
| Insulin (50 nM) | | 149 |
| Insulin (100 nM) | | 157 |
| Insulin (200 nM) | | 266 |
| Extract 1 | 10 | 158 |
| Extract 1 | 50 | 175 |
| Extract 1 | 250 | 156 |
| Extract 1 | 1000 | 157 |
| Extract 2 | 10 | 167 |
| Extract 2 | 50 | 159 |
| Extract 2 | 250 | 199 |
| Extract 2 | 1000 | 140 |
| Extract 3 | 10 | 236 |
| Extract 3 | 50 | 220 |
| Extract 3 | 250 | 200 |
| Extract 3 | 1000 | 230 |
| Extract 4 | 10 | 167 |
| Extract 4 | 50 | 162 |
| Extract 4 | 250 | 145 |
| Extract 4 | 1000 | 148 |
| Extract 5 | 10 | 139 |
| Extract 5 | 50 | 169 |
| Extract 5 | 250 | 202 |
| Extract 5 | 1000 | 808 |
| Extract 6 | 10 | 142 |
| Extract 6 | 50 | 295 |
| Extract 6 | 250 | 499 |
| Extract 6 | 1000 | 825 |
| Extract 7 | 10 | 128 |
| Extract 7 | 50 | 143 |
| Extract 7 | 250 | 136 |
| Extract 7 | 1000 | 455 |
| Extract 8 | 10 | 203 |
| Extract 8 | 50 | 185 |
| Extract 8 | 250 | 165 |
| Extract 8 | 1000 | 765 |
| Extract 9 | 10 | 163 |
| Extract 9 | 50 | 172 |
| Extract 9 | 250 | 213 |
| Extract 9 | 1000 | 332 |
| Extract 10 | 10 | 177 |
| Extract 10 | 50 | 208 |
| Extract 10 | 250 | 196 |
| Extract 10 | 1000 | 286 |
| Extract 11 | 50 | 252 |
| Extract 11 | 250 | 227 |
| Extract 11 | 1000 | 380 |
| Extract 11 | 2000 | 1379 |
| Extract 12 | 50 | 277 |
| Extract 13 | 250 | 291 |
| Extract 13 | 1000 | 213 |
| Extract 13 | 2000 | 502 |
| Extract 14 | 50 | 217 |
| Extract 14 | 250 | 270 |
| Extract 14 | 1000 | 1263 |
| Extract 14 | 2000 | 512 |
| Extract 15 | 50 | 196 |
| Extract 15 | 250 | 232 |
| Extract 15 | 1000 | 274 |
| Extract 15 | 2000 | 615 |

Table 2 summarizes the dose-dependent uptake of [1,2-³H]2-Deoxy-D-glucose (2-deoxyglucose) uptake in 3T3-L1 cells in the presence of SRB Extracts 1-10, and the dose-dependent uptake of 3-O-methylglucose in 3T3-L1 cells in the presence of extracts 11-14.2 shows. Data is shown as increase (stimulation) over Control and 200 nM insulin.

TABLE 2
Dose-dependent uptake of [1,2-³H]2-Deoxy-D-glucose (2-deoxyglucose)
uptake in 3T3-L1 cells in the presence of SRB Extracts 1-10, and
the dose-dependent uptake of 3-O-methylglucose in 3T3-L1 cells in
the presence of extracts 11-14 presented as maximum cpms.

| | Max | Increase over | Increase over 200 |
|---|---|---|---|
| Sample | CPM | Control | nM Insulin |
| Control | 131 | NA | 0.5 |
| 50 nM Insulin | 149 | 1.1 | 0.6 |
| 100 nM Insulin | 157 | 1.2 | 0.6 |
| 200 nM Insulin | 266 | 2.0 | NA |
| Extract 1 | 175 | 1.3 | 0.7 |
| Extract 2 | 199 | 1.5 | 0.7 |
| Extract 3 | 230 | 1.8 | 0.9 |
| Extract 4 | 167 | 1.3 | 0.6 |
| Extract 5 | 808 | 6.2 | 3.0 |
| Extract 6 | 825 | 6.3 | 3.1 |
| Extract 7 | 455 | 3.5 | 1.7 |
| Extract 8 | 765 | 5.8 | 2.9 |
| Extract 9 | 332 | 2.5 | 1.2 |
| Extract 10 | 286 | 2.2 | 1.1 |
| Extract 11 | 380 | 2.9 | 1.4 |
| Extract 12 | 291 | 2.2 | 1.1 |
| Extract 13 | 512 | 3.9 | 1.9 |
| Extract 14 | 274 | 2.1 | 1.0 |

Data is shown as increase (stimulation) over Control and 200 nM insulin.

Table 3 shows the known compounds in stabilized rice bran Extracts 1 to 14 that are inhibitors of glucose uptake. Specifically, Table 2 lists the chemical name, exact mass, range of relative abundances, and weight (μg) per 100 mg based on their relative abundances of these compounds in the SRB extracts. Compounds in SRB-DI that contribute to the glucose uptake activity include lipid soluble sterols and fatty acids, with the majority being fatty acids. Fatty acids, particularly arachidonic acid, have been shown to stimulate glucose uptake through cycoloxygenase-independent mechanisms by increasing GLUT1 and GLUT4 activity in plasma membranes (J. B. P. Claire Nugent, J. P. Whitehead, J. M. Wentworth, V. Krishna K. Chatterjee, and S. O'Rahilly, 2001. Arachidonic acid stimulates glucose uptake in 3T3-L1 adipocytes by increasing GLUT1 and GLUT4 levels at the plasma membrane. J. Biol. Chem. 278:9149-9157).

TABLE 3
Summary of compounds in SRB Extracts 1 to 14 identified
as active contributors to glucose uptake enhancement.

| | m/z | Relative | Wt per 100 mg |
|---|---|---|---|
| Compound Name | (M + H+) | Abundance (%) | (μg) |
| 2-Methyl-2-butenoic acid amide | 100.08 | 0.2-1.3 | 5-46 |
| 8-Methyl-8-azabicyclo[3.2.1]octane-3,6-diol | 158.12 | 0.1-19.4 | 8-408 |
| 4-Isopropyl-1,2-benzenediol di-methyl ether | 181.12 | 1.9-24.6 | 104-904 |
| Glutamine N 5-Isopropyl | 189.12 | 0.2-4.3 | 19-177 |
| 6,10,14-Trimethyl-5,9,13-pentadecatrien-2-one | 263.24 | 10.4-37.0 | 285-1903 |
| 11,14-Octadecadienal | 265.25 | 8.5-26.7 | 260-1385 |
| 9,11,13,15-Octadecatetraenoic acid | 277.22 | 4.1-21.1 | 292-1251 |
| 7-Hydroxy-14,15-dinor-8(17)-labden-13-one | 279.23 | 38.5-100.0 | 1059-8139 |
| 9,12-Octadecadienoic acid | 281.25 | 12.8-100.0 | 352-7625 |
| 10-Octadecenoic acid | 283.26 | 10.0-100.0 | 274-7852 |
| 16-Hydroxy-9,12,14-octadecatrienoic acid | 295.23 | 11.4-35.5 | 623-1611 |
| 13-Oxo-9-octadecenoic acid | 297.25 | 19.8-45.1 | 543-3091 |
| 4-Oxooctadecanoic acid | 299.26 | 7.2-17.0 | 211-877 |
| Palmidrol | 300.29 | 4.5-13.0 | 209-768 |
| Fortimicin | 321.22 | 0.7-4.0 | 35-79 |
| Loesenerine | 338.28 | 1.6-9.8 | 94-411 |
| 1,2-Dihydroxy-5-heneicosen-4-one | 341.30 | 2.0-7.4 | 108-242 |
| 2-Amino-4-octadecene-1,3-diol N-Ac | 342.30 | 1.2-11.8 | 64-368 |
| 2-(Aminomethyl)-2-propenoic acid N-Hexadecanoyl, Me | 354.29 | 1.9-10.4 | 105-364 |
| ester | | | |
| Glycerol 1-(9Z,12Z-octadecadienoate) | 355.29 | 6.4-29.7 | 228-1673 |
| CyclobuxophyllineO | 356.29 | 2.9-12.8 | 156-470 |
| Glycerol 1-(9Z-octadecenoate) | 357.30 | 6.3-32.6 | 241-2159 |
| Buxandonine L | 358.31 | 3.0-12.5 | 128-583 |
| 12-Hydroxy-25-nor-17-scalaren-24-al | 359.30 | 1.2-8.8 | 63-243 |
| ConioidineA | 366.31 | 1.9-7.8 | 63-236 |

In addition the identified MS peak value (m/z), relative abundances and weight per 100 mg of extract are provided.

### B. FABP4 Inhibition

Table 4 shows the results of FABP4 binding in Extracts 1 to 14. Extracts 1 to 8 were obtained from SRB feedstock A, while extracts 9 to 22 were obtained from SRB feedstock B. Table 5 lists the identified known compounds in stabilized rice bran extracts 1 to 14 that are inhibitors of FABP4. Table 5 provides the chemical name, exact mass, range of relative abundances, and weight (μg) per 100 mg based on their relative abundances of these compounds in the SRB extracts, as well as estimated IC₅₀ values.

TABLE 4
Summary of FABP4 inhibition by SRB extracts providing
the IC₅₀ values, the R² and N values for the bioassays.

| Extract | | IC₅₀ | | |
|---|---|---|---|---|
| No. | Extraction Conditions | (μg mL⁻¹ | R² | N |
| 1 | Rice Bran Ethanolic Extract by 80% ethanol | NA | NA | NA |
| | leaching from feedstock A at room | | | |
| | temperature | | | |
| 2 | Rice Bran Ethanolic Extract by Distilled | NA | NA | NA |
| | Water leaching from feedstock A at 40° C. | | | |
| 3 | Rice Bran Ethanolic Extract by 20% ethanol | NA | NA | NA |
| | leaching from feedstock A at 40° C. | | | |
| 4 | Rice Bran Ethanolic Extract by 40% ethanol | NA | NA | NA |
| | leaching from feedstock A at 40° C. | | | |
| 5 | Rice Bran Ethanolic Extract by 60% ethanol | 617.3 | 0.988 | 15 |
| | leaching from feedstock A at 40° C. | | | |
| 6 | Rice Bran Ethanolic Extract by 80% ethanol | 332.1 | 0.99 | 15 |
| | leaching from feedstock A at 40° C. | | | |
| 7 | Rice Bran Ethanolic Extract by ethanol | 642.4 | 0.975 | 15 |
| | leaching from HS01590 feedstock A at 40° C. | | | |
| 8 | Rice Ethanolic Extract by 80% ethanol | 298.0 | 0.949 | 15 |
| | leaching from feedstock A at 60° C. | | | |
| 9 | Rice Bran CO₂ extract by SFT at 40° C. and | 436.2 | 0.949 | 15 |
| | 300Bar on HS00332 | | | |
| 10 | Rice Bran CO₂ extract by SFT at 40° C. and | 517.4 | 0.958 | 15 |
| | 500 Bar on feedstock B | | | |
| 11 | Rice Bran CO₂ extract by SFT at 60° C. and | 313.6 | 0.984 | 15 |
| | 300 Bar on feedstock B | | | |
| 12 | Rice Bran CO₂ extract by SFT at 60° C. and | 558.4 | 0.937 | 15 |
| | 500Bar on feedstock B | | | |
| 13 | Rice Bran CO₂ extract by SFT at 80° C. and | 176.9 | 0.987 | 15 |
| | 300Bar on feedstock B | | | |
| 14 | Rice Bran CO₂ extract by SFT at 80° C. and | 349.2 | 0.965 | 15 |
| | 500Bar on feedstock B | | | |
| 15 | Rice Bran Ethanolic Extract by 80% ethanol | ND | 0.747 | 10 |
| | from feedstock B SFT residue at room | | | |
| | temperature | | | |
| 16 | Rice Bran Ethanolic Extract by Distilled | ND | 0.729 | 10 |
| | Water from feedstock B SFT residue at 40° C. | | | |
| 17 | Rice Bran Ethanolic Extract by 20% ethanol | ND | 0.493 | 10 |
| | from feedstock B SFT residue at 40° C. | | | |
| 18 | Rice Bran Ethanolic Extract by 40% ethanol | ND | 0.935 | 10 |
| | from feedstock B SFT residue at 40° C. | | | |
| 19 | Rice Bran Ethanolic Extract by 60% ethanol | ND | 0.77 | 10 |
| | from feedstock B SFT residue at 40° C. | | | |
| 20 | Rice Bran Ethanolic Extract by 80% ethanol | NA | NA | NA |
| | from feedstock B SFT residue at 40° C. | | | |
| 21 | Rice Bran Ethanolic Extract by ethanol from | ND | 0.947 | 10 |
| | feedstock B SFT residue at 40° C. | | | |
| 22 | Rice Bran Ethanolic Extract by 80% ethanol | NA | NA | NA |
| | from feedstock B SFT residue at 60° C. | | | |

TABLE 5
Summary of FABP4 inhibiting compounds in SRB Extracts 1 to 14.

| | | Relative | | |
|---|---|---|---|---|
| | Molecular | Abundance | Weight per | Predicted |
| Compound Name | Mass | (%) | 100 mg (μg) | IC₅₀ (μM) |
| 4,5-Dihydro-4-hydroxy-5-methyl-2- | 312.2670 | 3.64-42.41 | 169.98-1443.30 | 61.03-85.04 |
| tetradecyl-2(3H)-furanone | | | | |
| Pregnane-2,3,6-triol | 336.2709 | 2.33-43.18 | 132.59-1469.28 | 44.21-80.39 |
| 5-(8-Heptadecenyl)dihydro-3- | 338.2854 | 2.58-63.20 | 117.41-2150.71 | 38.91-116.97 |
| hydroxy-2(3H)-furanone | | | | |
| 24-Nor-4(23),9(11)-fernadiene | 394.3646 | 2.99-42.37 | 105.06-1313.12 | 29.87-61.26 |
| 24-Nor-12-ursene | 396.3766 | 0.49-100.00 | 61.53-3449.39 | 17.41-160.11 |
| 11,13(18)-0leanadiene | 408.3772 | 2.93-62.74 | 102.92-2004.82 | 28.26-90.32 |
| 14-Methyl-9,19-cycloergost-24(28)- | 412.3733 | 1.05-18.05 | 78.20-576.65 | 21.26-25.73 |
| en-3-ol | | | | |
| Montecristin | 574.4990 | 2.42-63.79 | 88.94-2170.87 | 17.36-69.52 |
| 3-(3,4-Dihydroxyphenyl)-2- | 600.5155 | 1.41-97.32 | 42.81-3311.85 | 7.99-101.47 |
| propenoicacid Triacontyl ester | | | | |
| Bombiprenone | 602.5337 | 1.37-78.19 | 37.40-2660.64 | 6.96-81.25 |
| Glycerol 1,2-dialkanoates; Glycerol | 616.5157 | 0.77-55.17 | 26.87-1877.32 | 4.89-56.03 |
| 1,2-di-(9 Z,12 Z-octadecadienoate) | | | | |

Compounds with their corresponding molecular mass, relative abundances, weight per 100 mg of extract and predicted IC₅₀ values (based on contribution across all actives).

Table 6 summarizes the active compounds in SRB Extract 6 providing the activity endpoint, the molecular mass, relative abundances, weight per 100 milligram of extract, and the predicted IC₅₀ value (based on contribution across all actives).

TABLE 6
Summary of active compounds in SRB Extract 6.

| | | | Relative | Wt per | |
|---|---|---|---|---|---|
| | | Molecular | Abundance | 100 mg | Predicted |
| Compound Name | Endpoint | Mass | (%) | (μg) | IC₅₀ (μM) |
| 2-Methyl-2-butenoic acid | Glucose | 99.074 | 1.26 | 20 | NA* |
| Amide | uptake | | | | |
| 8-Methyl-8- | Glucose | 157.110 | 19.36 | 300 | NA* |
| azabicyclo[3.2.1]octane-3,6- | uptake | | | | |
| diol | | | | | |
| 4-Isopropyl-1,2-benzenediol | Glucose | 180.104 | 24.63 | 380 | NA* |
| Di-Me ether | uptake | | | | |
| Glutamine N 5-Isopropyl | Glucose | 188.110 | 4.32 | 70 | NA* |
| | uptake | | | | |
| 6,10,14-Trimethyl-5,9,13- | Glucose | 262.230 | 22.85 | 360 | NA* |
| pentadecatrien-2-one | uptake | | | | |
| 11,14-Octadecadienal | Glucose | 264.245 | 13.66 | 210 | NA* |
| | uptake | | | | |
| 9,11,13,15- | Glucose | 276.210 | 21.11 | 330 | NA* |
| Octadecatetraenoic acid | uptake | | | | |
| 7-Hydroxy-14,15-dinor-8(17)- | Glucose | 278.225 | 100.00 | 1560 | NA* |
| labden-13-one | uptake | | | | |
| 9,12-Octadecadienoic acid | Glucose | 280.240 | 39.18 | 610 | NA* |
| | uptake | | | | |
| 10-Octadecenoic acid | Glucose | 282.255 | 20.43 | 320 | NA* |
| | uptake | | | | |
| 16-Hydroxy-9,12,14- | Glucose | 294.222 | 35.53 | 550 | NA* |
| octadecatrienoic acid | uptake | | | | |
| 13-Oxo-9-octadecenoic acid | Glucose | 296.239 | 38.16 | 590 | NA* |
| | uptake | | | | |
| 4-Oxooctadecanoic acid | Glucose | 298.256 | 11.09 | 170 | NA* |
| | uptake | | | | |
| Palmidrol | Glucose | 299.274 | 10.98 | 170 | NA* |
| | uptake | | | | |
| 4,5-Dihydro-4-hydroxy-5- | FABP4 | 312.267 | 12.24 | 190 | 73.32 |
| methyl-2-tetradecyl-2(3H)- | inhibition | | | | |
| furanone | | | | | |
| Fortimicin | Glucose | 320.204 | 4.03 | 60 | NA* |
| | uptake | | | | |
| Pregnane-2,3,6-triol | FABP4 | 336.271 | 12.47 | 190 | 69.39 |
| | inhibitor | | | | |
| Loesenerine | Glucose | 337.272 | 7.61 | 120 | NA* |
| | uptake | | | | |
| 5-(8-Heptadecenyl)dihydro-3- | FABP4 | 338.285 | 17.51 | 270 | 96.84 |
| hydroxy-2(3H)-furanone | inhibition | | | | |
| 1,2-Dihydroxy-5-heneicosen- | Glucose | 340.286 | 7.42 | 120 | NA* |
| 4-one | uptake | | | | |
| 2-Amino-4-octadecene-1,3- | Glucose | 341.290 | 11.80 | 180 | NA* |
| diol N—Ac | uptake | | | | |
| 2-(Aminomethyl)-2-propenoic | Glucose | 353.283 | 10.42 | 160 | NA* |
| acid N-Hexadecanoyl, Me | uptake | | | | |
| ester | | | | | |
| Glycerol 1-alkanoates | Glucose | 354.283 | 11.88 | 190 | NA* |
| Glycerol 1-(9Z,12Z- | uptake | | | | |
| octadecadienoate) | | | | | |
| Cyclobuxophylline O | Glucose | 355.280 | 11.95 | 190 | NA* |
| | uptake | | | | |
| Glycerol 1-alkanoates | Glucose | 356.295 | 12.64 | 200 | NA* |
| Glycerol 1-(9Z- | uptake | | | | |
| octadecenoate) | | | | | |
| Buxandonine L | Glucose | 357.292 | 12.50 | 190 | NA* |
| | uptake | | | | |
| 12-Hydroxy-25-nor-17- | Glucose | 358.284 | 4.35 | 70 | NA* |
| scalaren-24-al | uptake | | | | |
| ConioidineA | Glucose | 365.296 | 7.79 | 120 | NA* |
| | uptake | | | | |
| 24-Nor-4(23),9(11)- | Glucose | 394.363 | 21.38 | 330 | NA* |
| fernadiene | uptake | | | | |
| 24-Nor-12-ursene | FABP4 | 396.377 | 42.21 | 660 | 199.25 |
| | inhibition | | | | |
| 11,13(18)-Oleanadiene | FABP4 | 408.377 | 19.87 | 310 | 91.04 |
| | inhibition | | | | |
| 14-methyl-9,19-cycloergost- | FABP4 | 412.373 | 12.28 | 190 | 60.55 |
| 24(28)-en-3-ol | inhibition | | | | |
| Montecristin | FABP4 | 574.499 | 12.84 | 200 | 40.01 |
| | inhibition | | | | |
| 3-(3,4-Dihydroxyphenyl)-2- | FABP4 | 600.516 | 9.53 | 150 | 29.68 |
| propenoicacid Triacontyl | inhibition | | | | |
| ester | | | | | |
| Bombiprenone | FABP4 | 602.534 | 8.53 | 130 | 26.50 |
| | inhibition | | | | |
| Glycerol 1,2-dialkanoates; | FABP4 | 616.516 | 8.12 | 130 | 24.66 |
| Glycerol 1,2-di-(9Z,12Z- | inhibition | | | | |
| octadecadienoate) | | | | | |

*NA = IC₅₀ cannot be predicted

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A rice bran extract comprising at least one compound selected from the group consisting of 0.001 to 5% by weight of 2-methyl-butenoic acid, 0.001 to 5% by weight of 8-methyl-8-azabicyclo[3.2.1]octane-3,6-diol, 0.01 to 5% by weight of 4-isopropyl-1,2-benzenediol di-methyl ether, 0.005 to 5% by weight of glutamine N 5-isopropyl, 0.05 to 10% by weight of 6,10,14-trimethyl-5,9,13-pentadecatriene-2-one, 0.05 to 10% by weight of 11, 14 octadecadienal, 0.05 to 10% by weight of 9,11,13,15-octadecatetraenoic acid, 0.1 to 20% by weight of 7-hydroxy-14,14-dinor-8(17)-labden-13-one, 0.05 to 20% by weight of 9,12-octadecenoic acid, 0.05 to 20% by weight of 10-octadecenoic acid, 0.01 to 15% by weight of 16-hydroxy-9,12,14-octadecatrienoic acid, 0.05 to 15% by weight of 13-oxo-9-octadecenoic acid, 0.01 to 5% by weight of 4-oxooctadecenoic acid, 0.05 to 5% by weight of palmidrol, 0.005 to 5% by weight of fortimicin, 0.005 to 5% by weight of loeserinine, 0.01 to 5% by weight of 1,2-dihydroxy-5-heneicosen-4-one, 0.005 to 5% by weight of 2-amino-4-octadecene-1,3-diol, 0.01 to 5% by weight of 2-(aminomethyl)-2-propenoic acid N-hexadecanoyl methyl ester, 0.01 to 10% by weight of glycerol 1-alkanoates glycerol 1-octadecadienoate, 0.01 to 5% by weight of cyclobuxophylline O, 0.01 to 20% by weight of glycerol 1-alkanoates glycerol 1-octadecenoate, 0.01 to 5% by weight of buxandonine L, 0.005 to 5% by weight of 12-hydroxy-25-nor-17-scalarene-24-al, 0.005 to 5% by weight of coniodine A and 0.05 to 10% by weight of 24-nor-4(23),9(11)-fernidine.

2. The rice bran extract of claim 1, comprising at least one compound selected from the group consisting of 0.01 to 1% by weight of 2-methyl-butenoic acid, 0.01 to 2% by weight of 8-methyl-8-azabicyclo[3.2.1]octane-3,6-diol, 0.1 to 3% by weight of 4-isopropyl-1,2-benzenediol di-methyl ether, 0.01 to 1% by weight of glutamine N 5-isopropyl, 0.1 to 3% by weight of 6,10,14-trimethyl-5,9,13-pentadecatriene-2-one, 0.1 to 2% by weight of 11, 14 octadecadienal, 0.2 to 5% by weight of 9,11,13,15-octadecatetraenoic acid, 1 to 10% by weight of 7-hydroxy-14,14-dinor-8(17)-labden-13-one, 0.3 to 5% by weight of 9,12-octadecenoic acid, 0.2 to 5% by weight of 10-octadecenoic acid, 0.5 to 5% by weight of 16-hydroxy-9,12,14-octadecatrienoic acid, 0.5 to 5% by weight of 13-oxo-9-octadecenoic acid, 0.2 to 1% by weight of 4-oxooctadecenoic acid, 0.1 to 1% by weight of palmidrol, 0.01 to 0.5% by weight of fortimicin, 0.1 to 1% by weight of loeserinine, 0.1 to 1% by weight of 1,2-dihydroxy-5-heneicosen-4-one, 0.05 to 1% by weight of 2-amino-4-octadecene-1,3-diol, 0.1 to 1% by weight of 2-(aminomethyl)-2-propenoic acid N-hexadecanoyl methyl ester, 0.2 to 2% by weight of glycerol 1-alkanoates glycerol 1-octadecadienoate, 0.1 to 1% by weight of cyclobuxophylline O, 0.1 to 2% by weight of glycerol 1-alkanoates glycerol 1-octadecenoate, 0.1 to 1% by weight of buxandonine L, 0.05 to 0.5% by weight of 12-hydroxy-25-nor-17-scalarene-24-al, 0.05 to 1% by weight of coniodine A and 0.2 to 2% by weight of 24-nor-4(23),9(11)-fernidine.

3. A rice bran extract comprising at least one compound selected from the group consisting of 1 to 100 μg of 2-methyl-butenoic acid, 0.1 to 1000 μg of 8-methyl-8-azabicyclo[3.2.1]octane-3,6-diol, 10 to 2000 μg of 4-isopropyl-1,2-benzenediol di-methyl ether, 1 to 500 μg glutamine N 5-isopropyl, 100 to 2500 μg of 6,10,14-trimethyl-5,9,13-pentadecatriene-2-one, 100 to 2000 μg of 11, 14 octadecadienal, 100 to 2000 μg of 9,11,13,15-octadecatetraenoic acid, 500 to 15,000 μg of 7-hydroxy-14,14-dinor-8(17)-labden-13-one, 100 to 15,000 μg of 9,12-octadecenoic acid, 100 to 15,000 of 10-octadecenoic acid, 100 to 2500 μg of 16-hydroxy-9,12,14-octadecatrienoic acid, 100 to 5000 μg of 13-oxo-9-octadecenoic acid, 100 to 1500 μg of 4-oxooctadecenoic acid, 100 to 1500 μg of palmidrol, 5 to 200 of fortimicin, 20 to 1000 μg of loeserinine, 10 to 500 μg of 1,2-dihydroxy-5-heneicosen-4-one, 10 to 500 μg of 2-amino-4-octadecene-1,3-diol, 10 to 500 μg of 2-(aminomethyl)-2-propenoic acid N-hexadecanoyl methyl ester, 100 to 2500 μg 1-alkanoates glycerol 1-octadecadienoate, 10 to 1000 μg cyclobuxophylline O, 100 to 3000 μg of glycerol 1-alkanoates glycerol 1-octadecenoate, 50 to 1000 μg of buxandonine L, 10 to 500 μg of 12-hydroxy-25-nor-17-scalarene-24-al, 10 to 500 μg of coniodine A, and 100 to 2000 of 24-nor-4(23),9(11)-fernidine, per 100 mg of extract.

4. A rice bran extract comprising at least one compound selected from the group consisting of 0.01 to 10% by weight of 4,5-dihydro-4-hydroxy-5-methyl-2-tetradecyl-2(3H)-furanone, 0.01 to 10% by weight of pregnane-2,3,6-triol, 0.01 to 10% by weight of 5-(8-heptadecenyl)dihydro-3-hydroxy-2(3H)-furanone, 0.01 to 10% by weight of 24-nor-4(23),9(11)-femadine, 0.01 to 10% by weight of 24-nor-12-ursene, 0.01 to 10% by weight of 11,13(18)-oleanadiene, 0.01 to 5% by weight of 14-methyl-9,19-cycloergost-24(28)-en-3-ol, 0.01 to 10% by weight of montecristin, 0.01 to 10% by weight of 3-(3,4-dihydroxyphenyl)-2-propenoic acid triacontyl ester, 0.01 to 10% by weight of bombiprenone, and 0.001 to 10% by weight of glycerol 1,2-di-(9Z,12Z-octadecadienoate).

5. The rice bran extract of claim 4 comprising at least one compound selected from the group consisting of 0.1 to 2% by weight of 4,5-dihydro-4-hydroxy-5-methyl-2-tetradecyl-2(3H)-furanone, 0.1 to 2% by weight of pregnane-2,3,6-triol, 0.1 to 3% by weight of 5-(8-heptadecenyl)dihydro-3-hydroxy-2(3H)-furanone, 0.1 to 2% by weight of 24-nor-4(23),9(11)-fernadine, 0.5 to 5% by weight of 24-nor-12-ursene, 0.05 to 3% by weight of 11,13(18)-oleanadiene, 0.05 to 1% by weight of 14-methyl-9,19-cycloergost-24(28)-en-3-ol, 0.05 to 3% by weight of montecristin, 0.05 to 5% by weight of 3-(3,4-dihydroxyphenyl)-2-propenoic acid triacontyl ester, 0.01 to 10% by weight of bombiprenone, and 0.01 to 2% by weight of glycerol 1,2-di-(9Z,12Z-octadecadienoate).

6. A rice bran extract comprising at least one compound selected from the group consisting of 50 to 3000 μg of 4,5-dihydro-4-hydroxy-5-methyl-2-tetradecyl-2(3H)-furanone, 50 to 3000 μg of pregnane-2,3,6-triol, 50 to 3000 μg of 5-(8-heptadecenyl)dihydro-3-hydroxy-2(3H)-furanone, 50 to 2000 μg of 24-nor-4(23),9(11)-femadine, 10 to 5000 μg of 24-nor-12-ursene, 25 to 2500 μg of 11,13(18)-oleanadiene, 10 to 1000 μg of 14-methyl-9,19-cycloergost-24(28)-en-3-ol, 10 to 3000 μg of montecristin, 5 to 5000 μg of 3-(3,4-dihydroxyphenyl)-2-propenoic acid triacontyl ester, 5 to 5000 of bombiprenone, and 5 to 3000 μg of glycerol 1,2-di-(9Z,12Z-octadecadienoate), per 100 mg of extract.

7. The rice bran extract of claim 1 having a fraction comprising a Direct Analysis in Real Time (DART) mass spectrometry chromatogram of any of FIGS. 1 to 14.

8. The rice bran extract of claim 1, wherein the extract has a glucose uptake stimulation greater than a glucose uptake stimulation of 200 nM insulin.

9. The rice bran extract of claim 8, wherein the glucose uptake stimulation of the extract is 0.5 to 5 times greater than the glucose uptake stimulation of 200 nM insulin.

10. The rice bran extract of claim 9, wherein the glucose uptake stimulation of the extract is 0.5 to 3.5 times greater than the glucose uptake stimulation of 200 nM insulin.

11. The rice bran extract of claim 10, wherein the glucose uptake stimulation of the extract is 0.7 to 3.1 times greater than the glucose uptake stimulation of 200 nM insulin.

12. The rice bran extract of claim 8, wherein the glucose uptake stimulation of the extract is more than 3 times greater than the glucose uptake stimulation of 200 nM insulin

13. The rice bran extract of claim 8, wherein the glucose uptake stimulation of the extract is about 3 times greater than the glucose uptake stimulation of 200 nM insulin.

14. The rice bran extract of claim 1, wherein the extract has a glucose uptake stimulation greater than the glucose uptake stimulation of control.

15. The rice bran extract of claim 14, wherein the extract glucose uptake stimulation is more than 1 times greater than the glucose uptake stimulation of control.

16. The rice bran extract of claim 14, wherein the extract glucose uptake stimulation is 1 to 10 times greater than the glucose uptake stimulation of control.

17. The rice bran extract of claim 14, wherein the extract glucose uptake stimulation is 2 to 7 times greater than the glucose uptake stimulation of control.

18. The rice bran extract of claim 14, wherein the extract glucose uptake stimulation is about 6 times greater than the glucose uptake stimulation of control.

19. The rice bran extract of any one claim 1, wherein the extract has a glucose uptake stimulation of 100 to 3000 counts per minute (cpm).

20. The rice bran extract of claim 19, wherein the extract has a glucose uptake stimulation of 100 to 1000 cpm.

21. The rice bran extract of claim 19, wherein the concentration of the extract is 5 to 2000 μg/mL.

22. The rice bran extract of claim 21, wherein the concentration of extract is 10 to 1000 μg/mL.

23. The rice bran extract of claim 22, wherein the concentration of extract is 10, 50, 250 or 1000 μg/mL.

24. The rice bran extract of claim 1, wherein the extract has an IC₅₀ value for FABP4 inhibition of less than 2000 μg/mL.

25. The rice bran extract of claim 24, wherein the IC₅₀ value for FABP4 inhibition is from 25 to 2000 μg/mL.

26. The rice bran extract of claim 25, wherein the IC₅₀ value for FABP4 inhibition is from 25 to 1000 μg/mL.

27. The rice bran extract of claim 26, wherein the IC₅₀ value for FABP4 inhibition is from 25 to 500 μg/mL.

28. A rice bran extract prepared by a process comprising the following steps:
a) providing a stabilized rice bran feedstock, and
b) extracting the feedstock.

29. The extract of claim 28, wherein the extracting step is an aqueous ethanol extraction.

30. The extract of claim 28, wherein the extracting step is supercritical carbon dioxide extraction.

31. A pharmaceutical composition comprising a rice bran extract of claim 1.

32. The pharmaceutical composition of claim 31, which is formulated as a functional food, dietary supplement, powder or beverage.

33. A method of inhibiting glucose uptake comprising administering to a subject in need thereof an effective amount of the extract of claim 1.

34. A method if inhibiting FABP4 binding comprising administering to a subject in need thereof an effective amount of the extract of claim 1.

35. The method of claim 33, wherein the subject has hyperglycemia.

36. The method of claim 33, wherein the subject has diabetes.

37. The method of claim 36, wherein the subject has type 1 diabetes.

38. The method of claim 36, wherein the subject has type 2 diabetes.

39. The method of claim 36, wherein the subject suffers from obesity and related metabolic disorders.
